(19) United States
(12) Patent Application Publication
(10) Pub. No.: US 2006/0085063 A1
(43) Pub. Date: Apr. 20, 2006

(54) Nano- and micro-scale engineering of polymeric scaffolds for vascular tissue engineering

(76) Inventors: V. Prasad Shastri, Nashville, TN (US); Jay Sy, Nashville, TN (US)

Correspondence Address: NEEDLE & ROSENBERG, P.C., SUITE 1000, 999 PEACHTREE STREET, ATLANTA, GA, 30309-3915, US

(21) Appl. No.: 11/252,229

(22) Filed: Oct. 17, 2005

Related U.S. Application Data
(60) Provisional application No. 60/619,156, filed on Oct. 15, 2004.

(51) Int. Cl. A61F 2/06

(52) U.S. Cl. 623/1.41; 424/422; 623/1.49; 606/152; 425/174.8 R

(57) ABSTRACT

In one aspect, the invention relates to a synthetic conduit comprising a substantially tubular body comprising substantially circumferential polymer fibers; wherein the body has an exterior surface, an interior surface, and a lumen extending therethrough; and wherein the body has microscale features disposed at the interior surface. In a further aspect, cells can be optionally adhered to the exterior and/or interior surface of the substantially tubular body. In various aspects, the conduit can be used as a vascular prosthesis, a stent, or a nerve regeneration scaffold. Methods of preparing and implanting same are also provided. This abstract is intended as a scanning tool for purposes of searching in the particular art and is not intended to be limiting of the present invention.

### PRIORITY CLAIM

This application claims the benefit of U.S. Application No. 60/619,156, filed Oct. 15, 2004, which is hereby incorporated herein by reference in its entirety.

### BACKGROUND

A. Coronary Heart Disease

According to the American Heart Association over 13 million Americans are suffering from some form of heart related ailment. On an average, one in every 2.6 deaths in the United States is due to cardiovascular diseases. Of these, over 50%, or 1 in every 5 deaths can be attributed to coronary heart disease (CHD). The annual cost associated with treating CHD is estimated to be over $140 billion. Myocardial infarction (infarct), which results in congestive heart failure, occurs when heart tissue is starved for oxygen due to diseased state in the arteries. This accounts for nearly 40% of the annual deaths that can be attributed to CHD. A major factor in congestive heart failure is the clogging of arteries due to plaque buildup. When traditional approaches of mechanically addressing the problem such as balloon angioplasty and stenting fail, the diseased artery has to be replaced by a healthy graft, which is typically harvested from the iliac or femoral artery of the patient. The usage of autologous grafts is plagued by its own set of problems, which include dimensional mismatch between host site versus graft and a shortage of graft to satisfy multiple bypass procedures. This has promoted the exploration of synthetic and engineered biological substitutes for replacement of small diameter arteries (<4 mm in diameter).

B. Artery Anatomy

An artery is composed of three distinct zones of cells, each of which play a precise structural and biofunctional role in ensuring vessel patency and function. The outermost layer of the artery is called the adventitia and is composed of fibroblasts. The role of the fibroblasts is to promote and sustain micro-vessels ingrowth into the next layer, which is the medial zone, composed of smooth muscle cells (SMC). The SMC secrete elastin, the elastic protein that is responsible for the elastic properties of arteries, and a collagen extracellular framework. This elastin-collagen framework, in addition, confers the vessel wall with the necessary mechanical strength to survive the high radial pressures (upper limit 180 mm of Hg, 24 kPa) of arterial circulation. The SMC are circumferentially (parallel to the short axis) aligned, i.e., perpendicular to the arterial flow and this is critical to ensure the radial distensibility of the artery. The phenotype of the SMC is very important as well and is dependent on its shape. Ensuring the right SMC phenotype (α-actin positive, elastin positive) is important for the vasoactive characteristics of the artery. Therefore, any synthetic vascular graft design preferably incorporates means to achieve circumferential alignment of SMC.

The medial zone transitions into a smooth muscle intima that is responsible for supporting the natural antithrombogenic coating in the lumen of the blood vessel, namely the endothelial cells (EC). The antithrombogenic property of the EC layer is as a consequence of nitrous oxide production by these cells that prevent adhesion and activation of platelets, which is the first step toward clot formation. Therefore, engineering a synthetic or biological substitute for an artery poses several challenges among which vessel wall burst pressure and endothelial cell retention in the lumen are key. To date, two avenues have been explored toward development of small diameter vascular graft substitutes (1) synthetic polymer substitutes and (2) tissue engineered vascular grafts.

C. Vascular Occlusion and Treatment

Occlusion of arteries due to plaque deposition and thrombosis results in reduced blood flow. Depending on the arteries affected, occlusion may lead to peripheral vascular disease, stroke or angina pectoris/myocardial infarct, making it the single largest cause of death in the United States. Treatment options for restoring flow through occluded arteries include thrombolytic agents, mechanical means of opening lumens such as balloon angioplasty and stenting, as well as bypassing the blocked segment, which is frequently the preferred option. Brener, S. J., et al., Propensity analysis of long-term survival after surgical or percutaneous revascularization in patients with multivessel coronary artery disease and high-risk features, Circulation, 2004, 109(19): p. 2290-5. Typically, autografts are used to bypass the occluded artery although their harvest is associated with donor site morbidity and, in some cases, is precluded by lack of appropriate available donor vessels.

D. Synthetic Vascular Grafts

Synthetic polymeric grafts are potentially attractive options, as polymer supply and fabrication are not limiting factors. Expanded polytetrafluoroethylene (ePTFE) and Dacron are used clinically as grafts for large-diameter vessels (>6 mm) but when used for small-diameter grafts (<6 mm), intimal hyperplasia at the anastomoses, attributed to compliance mismatch between the graft and native vessel, and thrombus formation, attributed to the thrombogenecity of the synthetic polymers used, result in an unacceptable patency. Zilla, P. and H. Greisler, Tissue Engineering of Vascular Prosthetic Grafts, 1999, Austin: R.G. Landes Company. Use of polyurethanes (PU), which have compliance values closer to native tissue (see How, T. V. and R. M. Clarke, The elastic properties of a polyurethane arterial prosthesis, J Biomech, 1984. 17(8): p. 597-608.), may eliminate compliance mismatch associated with relatively rigid materials like PTFE and Dacron, but thrombus formation is still a concern.

E. Tissue Engineering

Tissue engineering is becoming a method of choice for the development of implants in surgery. However, to create three-dimensional scaffolds conducive for cell deposition and cell proliferation, the dynamic interaction of cell and matrix substances must be understood. Three-dimensional polymer matrix systems have shown considerable promise for tissue engineering because of their increased surface area for cell growth, pathways for cellular migration, and channels for transport of nutrients to cells. Pores in these structures can aid in the polymer resorption-graft incorporation cycle by increasing pathways through which cells can migrate, increasing the surface area for cell attachment, providing pathways by which nutrients may reach the cells, and increasing the polymer surface exposed to the degradation medium.

Despite advances in tissue engineering, current three-dimensional vascular scaffolds generally lack structure sufficient to achieve adequate cell attachment. Accordingly, there remains a need for three-dimensional vascular scaffolds having suitable properties.

### SUMMARY

In accordance with the purpose(s) of the invention, as embodied and broadly described herein, the invention, in one aspect, relates to a synthetic conduit comprising a substantially tubular body comprising substantially circumferential electrospun polymer fibers; wherein the body has an exterior surface, an interior surface, and a lumen extending therethrough; and wherein the body has microscale features disposed at the interior surface. Cells can be optionally adhered to the exterior and/or interior surface of the substantially tubular body.

In a further aspect, the invention relates to a synthetic conduit comprising a substantially tubular body comprising substantially circumferential polymer fibers; wherein the body has an exterior surface, an interior surface, and a lumen extending therethrough; and wherein the body has microscale features disposed at the interior surface. Cells can be optionally adhered to the exterior and/or interior surface of the substantially tubular body.

In a further aspect, the invention relates to multilayered conduits.

In a further aspect, the invention relates to a vascular prosthesis comprising a substantially tubular body comprising substantially aligned, substantially circumferential, electrospun polyurethane fibers; wherein the body has an exterior surface, an interior surface, and a lumen with a diameter of from about 2 mm to about 4 mm extending therethrough; wherein the body has microscale grooves disposed at the interior surface substantially parallel to the lumen; wherein the grooves have an average width of from about 50 μm to about 100 μm and an average depth of from about 20 μm to about 60 μm; wherein at least one smooth muscle cell is adhered to the exterior surface; and wherein at least one endothelial cell is adhered to the interior surface.

In a further aspect, the invention relates to a nerve regeneration scaffold comprising a substantially tubular body comprising substantially aligned, substantially circumferential, electrospun polyurethane fibers; wherein the body has an exterior surface, an interior surface, and a lumen with a diameter of from about 2 mm to about 4 mm extending therethrough; wherein the body has microscale ridges or grooves disposed at the interior surface substantially parallel to the lumen; and wherein at least one transfected fibroblast cell is adhered to the exterior surface. Cells can be optionally adhered to the interior surface of the substantially tubular body.

In a further aspect, the invention relates to a method of preparing a synthetic conduit comprising the step of electrospinning a solution of polymer onto a rotating mandrel bearing microscale features, thereby providing a substantially tubular body comprising substantially circumferential electrospun polymer fibers; wherein the body has an exterior surface, an interior surface, and a lumen extending therethrough; and wherein the body has complementary microscale features disposed at the interior surface. Cells can be optionally adhered to the exterior and/or interior surface of the substantially tubular body.

In a further aspect, the invention relates to a method of preparing a synthetic conduit comprising the step of spinning a polymer onto a rotating mandrel bearing microscale features, thereby providing a substantially tubular body comprising substantially circumferential polymer fibers; wherein the body has an exterior surface, an interior surface, and a lumen extending therethrough; and wherein the body has complementary microscale features disposed at the interior surface. Cells can be optionally adhered to the exterior and/or interior surface of the substantially tubular body.

In a further aspect, the invention relates to a method of preparing a vascular prosthesis comprising the steps of: electrospinning a solution of polymer onto a rotating mandrel bearing microscale features, thereby providing a substantially tubular body comprising substantially circumferential electrospun polymer fibers; wherein the body has an exterior surface, an interior surface, and a lumen extending therethrough; and wherein the body has complementary microscale features disposed at the interior surface; adhering at least one smooth muscle cell to the exterior surface; and adhering at least one endothelial cell to the interior surface.

In a further aspect, the invention relates to a method of implanting a vascular prosthesis comprising the steps of: providing the prosthesis produced by the methods of the invention; and implanting the prosthesis into a subject.

In a further aspect, the invention relates to a method of preparing a nerve regeneration scaffold comprising the steps of: electrospinning a solution of polymer onto a rotating mandrel bearing microscale features, thereby providing a substantially tubular body comprising substantially circumferential electrospun polymer fibers; wherein the body has an exterior surface, an interior surface, and a lumen extending therethrough; and wherein the body has complementary microscale features disposed at the interior surface; and adhering at least one transfected fibroblast to the exterior surface. Cells can be optionally adhered to the exterior interior surface of the substantially tubular body.

In a further aspect, the invention relates to a method of implanting a nerve regeneration scaffold comprising the steps of: providing the scaffold produced by the methods of the invention; and implanting the scaffold into a subject.

In a further aspect, the invention relates to the products produced by the methods of the invention.

In an even further aspect, the invention relates to a biocompatible tubular prosthesis, comprising a matrix of highly aligned polymeric fibers having a tubular structure, proximal and distal ends, and a lumen extending therethrough, wherein the tubular structure has an exterior surface and an interior surface, and wherein the interior surface has reservoirs disposed at the interior surface. In one aspect, reservoirs can be disposed substantially perpendicular to the length of the lumen. In a further aspect, the reservoirs can be disposed substantially longitudinal to the length of the lumen. In a yet further aspect, smooth muscle cells can be in contact with the exterior surface of the tubular structure. In a still further aspect, endothelial cells can be in contact with the interior surface of the tubular structure. In a further aspect, the endothelial cells can be positioned in the reservoirs.

### BRIEF DESCRIPTION OF THE FIGURES

The accompanying drawings, which are incorporated in and constitute a part of this specification, illustrate several aspects and together with the description illustrate the disclosed compositions and methods.

FIG. 1 shows a schematic representation of vascular graft design.

FIG. 2 shows a schematic of the electrospinning process.

FIG. 3 shows the effect of water phase in the emulsion on fiber diameter and morphologies of fibers at various compositions.

FIG. 4 shows an electrospinning apparatus. Mandrel is rotated and translated laterally to collect oriented polymer nanofiber. Short tip-to-target distances allow for less fiber whipping and greater alignment.

FIG. 5 shows an electrospinning setup for spinning tubular scaffolds. The mandrel is mounted on a stage that enables side-by-side motion so that uniform fiber deposition can occur through the length of the scaffold.

FIG. 6 shows a dual-perfusion bioreactor for culturing and mechanical stimulation 10 of engineered artery. The white structure in the middle is a PU graft produced using the ES process.

FIG. 7 shows an electrospun fiber diameter versus percent aqueous phase curve.

FIG. 8 shows the effect of increasing aqueous content of the solution on the viscosity of the electrospinning solution; top: varying rotational speed; bottom: constant rotational speed.

FIG. 9 shows the effect of rotation speed on the viscosity of the electrospinning solution

FIG. 10 shows a proposed mechanism of emulsion stability.

FIG. 11 shows: Upper left, scanning electron micrograph of electrospun PU at 5000×. Upper right, electrospun collagen (40 mg/ml in 1,1,1,3,3,3-hexafluoro 2-propanol) at 5000×. Lower left, cospun collagen and PU fibers at 5000×. Lower right, zoom of cospun collagen and PU fibers at 20,000×.

FIG. 12 shows a normalized optical density vs. percent collagen. Colorimetric comparison of collagen composition of electrospun scaffold. Samples with varying amounts of collagen were stained with Sirius red. Bound dye was solublized in a basic solution and concentration determined spectrophotometrically.

FIG. 13 shows a scanning electron micrograph of aligned polyurethane fibers collected using custom electrospinning apparatus.

FIG. 14 shows stress vs. strain curves for a random sheet of PU nanofiber, circumferential direction of the aligned PU nanofiber scaffold, and longitudinal direction of the aligned PU scaffold.

FIG. 15 shows a segment of tubular scaffolds with a textured lumen and aligned exterior.

FIG. 16 shows textured sheets of PU fiber. Left panel, the alternating pattern of a wire mesh is retained on the sheet surface. Right panel, grid pattern of polymer mesh is retained, for example as a texture of peaks (e.g., protrusions, ridges, or plateaus) and valleys (e.g., reservoirs, wells, or grooves) on the sheet surface.

FIG. 17 shows: Left panel: (A) Silicon master, (B) Array of channels on PU film, & (C) Cross-section showing plateau (P) and floor of the channels (C); Right panel: Schematic of flow experiment.

FIG. 18 shows: Top panel: Distribution of bovine aortic endothelial cells (BAEC) (cell density, cells/mm²) after exposure to flow; Bottom panel: Left: Phase contrast image of PU film surface showing plateaus and channels and Right: Fluorescent image of the same region (white dots are cells stained with DAPI nuclear stain).

FIG. 19 shows a computational fluid dynamics simulation of the effects of changing channel depth (top panel) or changing channel width (bottom panel). Increasing the depth of the channel by 25% (gray line and bar, top panel) from the actual geometry used for endothelial retention studies (black line and bar, top panel) had the effect of increasing the wall shear stress in the plateau region, but decreasing the wall shear stress throughout the wall and channel. Decreasing the depth by a similar amount (dashed line and bar, top panel) had an opposite effect. Increasing the width of the channel by 10 μm (gray line and bar, bottom panel) from the actual geometry used in endothelial retention studies (black line and bar, bottom panel) increased the shear stress throughout the channel, without affecting the shear stress in the plateau regions. Similarly decreasing the width by 10 μm (dashed line and bar, bottom panel) had an opposite effect in the channel region, without affecting the plateau region.

FIG. 20 shows geometries (not drawn to scale) (A) and arrangement of micro-topography in the vascular graft luminal wall (B).

FIG. 21 shows a schematic of the slide assembly (Panel A) as well as the flow chamber and perfusion system (Panel B). Schematic is not drawn to scale.

FIG. 22 shows a computational fluid dynamics simulation of velocity profile and wall shear stress for micro-patterned surfaces corresponding to those used for endothelial retention studies. For the regions relatively far from the polyurethane surface, the velocity varied linearly with vertical distance (Panel A). Closer to the polyurethane surface, the velocity profile was altered by the presence of the channel (Panels B and C) resulting in reduced wall shear stress within the channel, with increased wall shear stress in the plateau region (Panel D, solid line). Note the average wall shear stress for the patterned surface (61 dyne/cm²) was nearly identical to the wall-shear stress calculated for an un-patterned surface with the same bulk flow (Panel D, dashed line).

FIG. 23 shows scanning electron micrographs of etched silicon wafer (Panel A) and cast polyurethane films shown en face (Panel B) or cut in cross-section (Panel C). The direction of flow is parallel to the long channels shown in Panel B. Plateaus and Channels are indicated with P and C, respectively.

FIG. 24 shows cell numbers normalized by total projected area for patterned and un-patterned surfaces. For patterned PU, the number of cells was counted separately for the channels and the plateaus and normalized by the sum of project areas of both the channels and plateaus, thus the combined height of the stacked shaded bars gives the total cells density in 1 mm² of patterned surface. Flow cultures were exposed to flow with an average wall shear stress of 60 dynes/cm² for 1 hour. Error bars represent standard deviation. For upper of the two stacked bars (i.e., to the cells on the plateaus), the negative error bars represent the error associated with the cells on the plateaus and the positive error bars represent the error associated with the sum of the cells on the plateaus and the cells in the channels. An asterisk indicates that the total number of cells on a patterned on un-patterned surface is significantly greater (α<0.05) than that of the un-patterned surface exposed to flow. No other differences in total cell numbers approach significance (α>0.05).

FIG. 25 shows phase contrast (left panel) and fluorescent (right panel) micrographs of patterned surfaces that were seeded with cells and exposed to 60 dynes/cm² for 1 hour. Channels (C) and Plateaus (P) are visible in light micrographs. Scale bars are 200 microns. The focal plane for acquisition was halfway between the channel bottom and upper edge, to ensure visualization of cells throughout the depth of the channel.

FIG. 26 shows a computational fluid dynamics simulation of the effects of changing channel depth (Panel A and C) or changing channel width (Panels B and D): Increasing the depth of the channel by 25% (gray line and bar, Panel A & C) from the actual geometry used for endothelial retention studies (black line and bar, Panel A & C) had the effect of increasing the wall shear stress in the plateau region, but decreasing the wall shear stress throughout the wall and channel. Decreasing the depth by a similar amount (dashed line and bar, Panel A & C) had an opposite effect. Increasing the width of the channel by 10 μm (gray line and bar, Panel B & D) from the actual geometry used in endothelial retention studies (black line and bar, Panel B & D) increased the shear stress throughout the channel, without affecting the shear stress in the plateau regions. Similarly decreasing the width by 10 μm (dashed line and bar, Panel B & D) had an opposite effect in the channel region, without affecting the plateau region.

### DETAILED DESCRIPTION

Before the present compounds, compositions, articles, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting.

### A. DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As used in the specification and the appended claims, the singular forms “a,” “an” and “the” include plural referents unless the context clearly dictates otherwise. Thus, for example, reference to “a component,” “a polymer,” or “an additive” includes mixtures of two or more such components, polymers, or additives, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data is provided in a number of different formats and that this data represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance may or may not occur, and that the description includes instances where said event or circumstance occurs and instances where it does not.

As used herein, the term “copolymer” means a polymer formed from two or more polymers. By way of example and without limitation, a copolymer can be an alternating copolymer, a random copolymer, a block copolymer, or a graft copolymer. A copolymer can be a segmented polymer.

As used herein, the term “segmented polymer” refers to a polymer having two or more chemically different sections of a polymer backbone that provide separate and distinct properties. These two sections may or may not phase separate. A “crystalline” material is one that has ordered domains (i.e., aligned molecules in a closely packed matrix), as evidenced by Differential Scanning Calorimetry, without a mechanical force being applied. A “noncrystalline” material is one that is amorphous at ambient temperature. A “crystallizing” material is one that forms ordered domains without a mechanical force being applied. A “noncrystallizing” material is one that forms amorphous domains and/or glassy domains in the polymer at ambient temperature.

As used herein, the term “biomaterial” refers to a material that is substantially insoluble in body fluids and tissues and that is designed and constructed to be placed in or onto the body or to contact fluid or tissue of the body. Ideally, a biomaterial will not induce undesirable reactions in the body such as blood clotting, tissue death, tumor formation, allergic reaction, foreign body reaction (rejection) or inflammatory reaction; will have the physical properties such as strength, elasticity, permeability and flexibility required to function for the intended purpose; can be purified, fabricated and sterilized easily; and will substantially maintain its physical properties and function during the time that it remains implanted in or in contact with the body. Biomaterials can also include both degradable and nondegradable polymers.

As used herein, a “medical device” can be defined as a device that has surfaces that contact blood or other bodily fluids in the course of their operation, which fluids are subsequently used in patients. This can include, for example, extracorporeal devices for use in surgery such as blood oxygenators, blood pumps, blood sensors, tubing used to carry blood and the like which contact blood which is then returned to the patient. This can also include endoprostheses implanted in blood contact in a human or animal body such as vascular grafts, stents, stent grafts, medical electrical leads, indwelling catheters, heart valves, and the like, that are implanted in blood vessels or in the heart. This can also include devices for temporary intravascular use such as catheters, guide wires, balloons, and the like which are placed into the blood vessels or the heart for purposes of monitoring or repair.

As used herein, the term “bioactive agent” means an agent that is capable of providing a local or systemic biological, physiological, or therapeutic effect in the biological system to which it is applied. For example, the bioactive agent can act to control infection or inflammation, enhance cell growth and tissue regeneration, control tumor growth, act as an analgesic, promote anti-cell attachment, and enhance bone growth, among other functions. Other suitable bioactive agents can include anti-viral agents, hormones, antibodies, or therapeutic proteins. Other bioactive agents include prodrugs, which are agents that are not biologically active when administered but, upon administration to a subject are converted to bioactive agents through metabolism or some other mechanism. Additionally, any of the compositions of the invention can contain combinations of two or more bioactive agents.

As used herein, the term “subject” means any target of administration. The subject can be an animal, for example, a mammal. In a further example, the subject can be a human.

As used herein, the terms “implanting” or “implantation” refer to any method of introducing a medical device, for example a vascular prosthesis, a stent, or a nerve regeneration scaffold, into a subject. Such methods are well known to those skilled in the art and include, but are not limited to, surgical implantation or endoscopic implantation. The term can include both sutured and bound implantation.

As used herein, the term “pharmaceutically active agent” includes a “drug” or a “vaccine” and means a molecule, group of molecules, complex or substance administered to an organism for diagnostic, therapeutic, preventative medical, or veterinary purposes. This term include externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA), ribonucleic acid (RNA), or mixtures or combinations thereof, including, for example, DNA nanoplexes. Pharmaceutically active agents include the herein disclosed categories and specific examples. It is not intended that the category be limited by the specific examples. Those of ordinary skill in the art will recognize also numerous other compounds that fall within the categories and that are useful according to the invention. Examples include a radiosensitizer, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifingal agent, a vaccine, a protein, or a nucleic acid. In a further aspect, the pharmaceutically active agent can be coumarin, albumin, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetominophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists such as clonidine; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; antiarrhythmic agents such as quinidine, lidocaine, tocainide hydrochloride, mexiletine hydrochloride, digoxin, verapamil hydrochloride, propafenone hydrochloride, flecainide acetate, procainamide hydrochloride, moricizine hydrochloride, and disopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hdyrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific aspect or combination of aspects of the disclosed methods.

It is understood that the compositions disclosed herein have certain functions. Disclosed herein are certain structural requirements for performing the disclosed functions, and it is understood that there are a variety of structures which can perform the same function which are related to the disclosed structures, and that these structures will typically achieve the same result.

### B. COMBINED NANO- AND MICRO-SCALE ENGINEERING

In one aspect, the invention combines the nano-scale engineering of polymeric fibers with micro-scale patterning of surfaces to design and fabricate superior synthetic conduits, for example vascular grafts, stents, or nerve regeneration scaffolds, derived of biocompatible polymers, for example biomedical grade polyurethane (PU) that offer enhanced biomaterial-cell interaction and functional outcomes, using a spinning, for example electrospinning, construction methodology.

Such a layer-by-layer approach allows for the tailoring of each surface of the scaffold to present specific information to the attached cells. It can be desirable to retain a layer of native endothelial cells in the lumen of a small diameter vascular graft as a natural anti-thrombogenic layer to prevent blood clotting and occlusions. In one aspect, controlling the surface topology of the lumen allows retaining greater numbers of endothelial cells under physiological levels of fluid flow through the graft. In a further aspect, on the outer surface of the graft, fiber alignment allows for the alignment of smooth muscle cells (SMC) along the direction of the fiber (circumferentially). This cellular alignment can allow for the coordinated response of SMCs to vasoactive substances. The ability for the graft to exhibit vasoactivity improves long-term graft survivability.

In a further aspect, the invention can comprise two general components: (1) a synthetic polymer conduit or scaffold which is relatively nonbiodegradable in nature and can form the core of an engineered vascular graft, whose function is to provide direction cues to smooth muscle cells (SMC), provide avenues for retention of endothelial cells (EC) in the lumen, and ensure superior mechanical properties at the outset; and (2) a biological component consisting of an outer zone composed of multiple layers of SMC and a lumen with a contiguous endothelium.

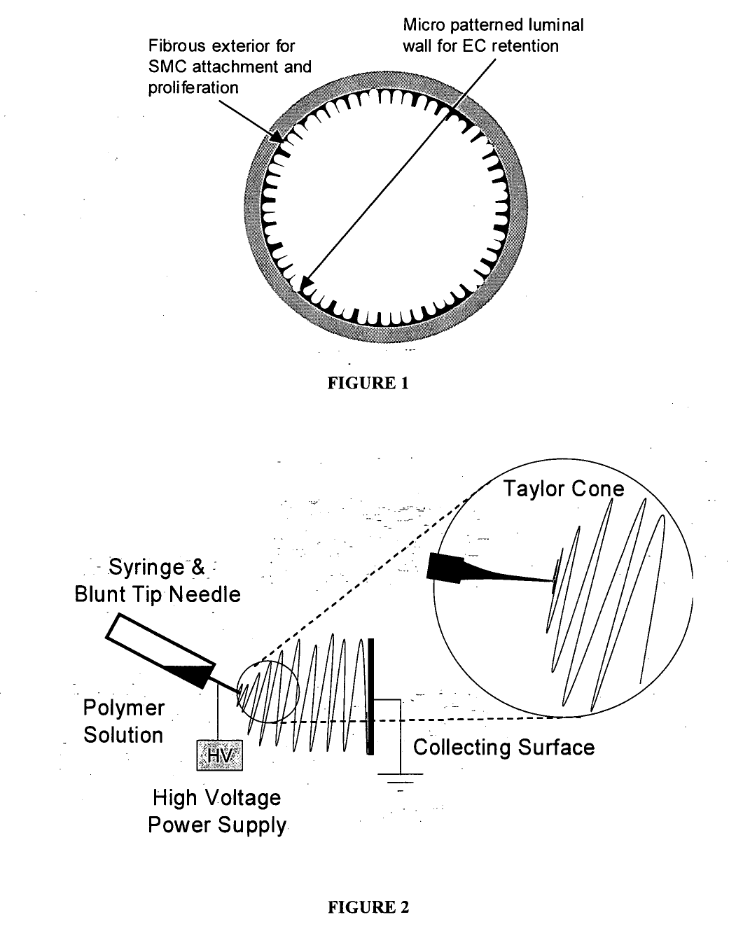

The synthetic conduit can be used as, for example, a vascular graft. In one aspect, such a graft can have a fibrous exterior composed of PU fibrous matrix in which the fibers exhibit substantially circumferential alignment throughout the length of the graft and a luminal surface comprising an array of well-defined micron scale recessed regions (FIG. 1). This design strategy can offer several advantages over both purely synthetic and purely biological systems. By physically separating the SMC intimal layer from the endothelium, seeding of the graft with SMC and EC can occur in a concurrent manner and thus both the SMC layer and evolving endothelium can be conditioned to flow in simultaneous fashion, thereby maximizing the role of hydrodynamic stimulation in the evolution of the micro-patterned luminal wall for EC retention. Further, since the vascular graft comprises, in one aspect, a central compliant zone of a permanent material, i.e., the polyurethane (PU) conduit or scaffold, higher burst strength can be achieved sooner, thus reducing graft maturation time, which is typically in 8-12 weeks for conventional strategies. The decreased maturation time reduces the cost associated with engineering of arterial graft substitutes. The physical separation of the endothelium from the exterior structure of graft can also ensure that neo-intimal hyperplasia will not occur, which is a potential problem with all purely biological grafts.

### C. SYNTHETIC CONDUITS

In one aspect, the invention relates to a synthetic conduit. In further aspects, the conduit can comprise a vascular prosthesis, a stent, or a nerve regeneration scaffold.

In a further aspect, the synthetic conduit of the invention can comprise a substantially tubular body comprising substantially circumferential electrospun polymer fibers; wherein the body has an exterior surface, an interior surface, and a lumen extending therethrough; and wherein the body has microscale features disposed at the interior surface.

In a further aspect, the synthetic conduit of the invention can comprise a substantially tubular body comprising substantially circumferential polymer fibers; wherein the body has an exterior surface, an interior surface, and a lumen extending therethrough; and wherein the body has microscale features disposed at the interior surface.

1. Substantially Tubular Body

In one aspect, the conduit can comprise a substantially tubular body. That is, the cross-section of the body can be, for example without limitation, round, substantially round, oval, substantially oval, elliptical, substantially elliptical, triangular, substantially triangular, square, substantially square, hexagonal, substantially hexagonal, or the like.

In one aspect, the tubular body can be provided having any length desired. In a further aspect, the length of the tubular body can be from about 1 cm to about 10 cm. For example, the length can be from about 2 cm to about 8 cm, from about 3 cm to about 7 cm, from about 4 cm to about 6 cm, about 1 cm, about 2 cm, about 3 cm, about 4 cm, about 5 cm, about 6 cm, about 7 cm, about 8 cm, about 9 cm, or about 10 cm. In a further aspect, the length can be greater than about 10 cm. In a further aspect, the length can be less than about 1 cm.

In one aspect, the tubular body can be provided having a wall thickness of from about 100 μm to about 1,000 μm. For example, the wall thickness can be of from about 100 μm to about 500 μm, from about 500 μm to about 1,000 μm, from about 200 μm to about 300 μm, from about 600 μm to about 800 μm, from about 200 μm to about 800 μm, from about 300 μm to about 700 μm, from about 400 μm to about 600 μm, or about 500 μm. In a further aspect, the wall thickness can be greater than about 1,000 μm. In a further aspect, the wall thickness can be less than about 100 μm.

2. Lumen

In one aspect, the tubular body can have a lumen extending therethrough. In one aspect, the lumen has the same cross-section as that of the tubular body. In a further aspect, the lumen has a different cross-section than that of the tubular body. For example, the cross-section of the lumen can be round, substantially round, oval, substantially oval, elliptical, substantially elliptical, triangular, substantially triangular, square, substantially square, hexagonal, substantially hexagonal, or the like.

In one aspect, the lumen has a diameter. In a further aspect, the diameter can be approximately the same as the diameter of the rotating mandrel used in the preparation of the substantially tubular body. It is understood that the diameter can vary along the length of the lumen. In a further aspect, the diameter can be from about 1 mm to about 5 mm. For example, the diameter can be from about 1 mm to about 5 mm, from about 1 mm to about 3 mm, from about 3 mm to about 5 mm, from about 2 mm to about 4 mm, about 1 mm, about 2 mm, about 3 mm, about 4 mm, or about 5 mm. In a further aspect, the diameter can be greater than about 5 mm. In a further aspect, the diameter can be less than about 1 mm. In an even further aspect, the diameter of less than about 20 mm, for example, less than about 10 mm, or less than about 5 mm.

In one aspect, the lumen has approximately the same length that of the tubular body.

3. Polymers

In one aspect, the conduit comprises substantially circumferential polymer fibers. In a further aspect, the conduit comprises substantially circumferential electrospun polymer fibers.

In one aspect, in substantially circumferential polymer fibers, the length of the polymer fibers is aligned substantially perpendicular to the length of the tubular body. Further, in one aspect, the polymer fibers are aligned substantially with the circumference of the cross-section of the tubular body. That is, the fibers are “wrapped” in a substantially spiral and overlapping position to comprise the tubular body. It is understood that a portion of the polymer fibers can be positioned other than substantially circumferentially.

In an even further aspect, the polymer fibers are biodegradable and the conduit can undergo controlled biodegradation occurring concomitantly with remodeling by the cells of a host subject. In this aspect, the conduit or scaffold thus functions as a substitute body part, and, while still functioning as a substitute body part, it functions as a remodeling template for the in-growth of host cells. In another aspect, the polymer fibers are nonbiodegradable, and the conduit or scaffold does not undergo biodegradation. In a further aspect, the polymer fibers are biocompatible.

In one aspect, the polymer fibers can comprise any biocompatible polymer known to those of skill in the art. In a further aspect, the polymer fibers comprise poly(lactic acid), poly(glycolic acid), or poly(ε-caprolactone), or a copolymer thereof, or a mixture thereof. In a further aspect, the polymer of the fibers can be polyethylene and/or polyurethane. In a further aspect, a polymer can be poly(lactide-co-glycolide), poly(lactic acid), poly(glycolic acid), poly(glaxanone), poly(orthoesters), poly(pyrolic acid), and poly(phosphazenes). Additional polymers that can be used include, but are not limited to, polyalkylene polymers and copolymers, fluorocarbon polymers and copolymers, polyester polymers and copolymers, polyether polymers and copolymers, silicone polymers and copolymers, and polyurethane polymers and copolymers. Other polymers that can be used include, but are not limited to, polyethylenes, polypropylenes, polytetrafluoroethylenes, poly(tetrafluoroethylene-co-hexafluoropropenes), modified ethylene-tetrafluoroethylene copolymers, ethylene chlorotrifluoroethylene copolymers, polyvinylidene fluorides, polyethylene oxides, polyethylene terephthalates, silicones, polyurethanes, polyether block amides, and polyether esters. In a further aspect, the polymer can be one or more polymers, for example, polypyrrole, polyaniline, polythiophene, poly(p-phenylene vinylene), polyparalene, or a mixture thereof. In a further aspect, the polymer can be poly (ethylene-vinyl acetate).

In one aspect, the polymer fibers comprise polyurethane fibers. Such polyurethanes include aliphatic as well as aromatic polyurethanes. In one aspect, useful polyurethanes include aromatic polyether polyurethanes, aliphatic polyether polyurethanes, aromatic polyester polyurethanes, aliphatic polyester polyurethanes, aromatic polycaprolactam polyurethanes, and aliphatic polycaprolactam polyurethanes. In a further aspect, useful polyurethanes include aromatic polyether polyurethanes, aliphatic polyether polyurethanes, aromatic polyester polyurethanes, and aliphatic polyester polyurethanes.

In a further aspect, the polymer fibers comprise segmented polyurethane fibers, for example, a poly(ether-urethane), a poly(ester-urethane), a poly(urea-urethane), a poly(carbonate-urethane), or mixture thereof. In a further aspect, the polymer fibers can be one or more degradable polyurethanes derived from glycerol and sebacic acid. See Wang Y., Ameer G. A., Sheppard B. J., Langer R., A tough biodegradable elastomer, Nature Biotechnology, 2002, 20(6):602-606. In a further aspect, the polymer fibers comprise medical grade and/or FDA-approved polyurethane fibers.

The chemistry of polyurethanes is extensive and well developed. Typically, polyurethanes are made by a process in which a polyisocyanate is reacted with a molecule having at least two hydrogen atoms reactive with the polyisocyanate, such as a polyol. That is, the polyurethane can be the reaction product of the following components: (A) a polyisocyanate having at least two isocyanate (—NCO) functionalities per molecule with (B) at least one isocyanate reactive group, such as a polyol having at least two hydroxy groups or an amine. Suitable polyisocyanates include diisocyanate monomers, and oligomers. The resulting polymer can be further reacted with a chain extender, such as a diol or diamine, for example. The polyol or polyamine can be a polyester, polyether, or polycarbonate polyol, or polyamine, for example.

Polyurethanes can be tailored to produce a range of products from soft and flexible to hard and rigid. They can be extruded, injection molded, compression molded, and solution spun, for example. Thus, polyurethanes can be important biomedical polymers, and are used in implantable devices such as artificial hearts, cardiovascular catheters, pacemaker lead insulation, etc.

In one aspect, the polymer fibers comprise a commercially available polyurethane useable for implantable applications. Commercially available polyurethanes used for implantable applications include ST1882 segmented polyether aromatic polyurethanes available from Stevens Urethane, Easthampton, Mass.; BIOSPAN® segmented polyurethanes available from Polymer Technology Group of Berkeley, Calif.; PELLETHANE® segmented polyurethanes available from Dow Chemical, Midland, Mich.; and TECOFLEX® and TECOFLEX® segmented polyurethanes available from Thermedics, Inc., Woburn, Mass. These polyurethanes and others are described in the article “Biomedical Uses of Polyurethanes,” by Coury et al., in Advances in Urethane Science and Technology, 9, 130-168, eds. K. C. Frisch and D. Klempner, Technomic Publishing Co., Lancaster, Pa. (1984). Typically, polyether polyurethanes exhibit more biostability than polyester polyurethanes, and are therefore generally preferred polymers for use in biological applications.

Polyether polyurethane elastomers, such as PELLETHANE® 2363-80A (P80A) and 2363-55D (P55D), which can be prepared from polytetramethylene ether glycol (PTMEG) and methylene bis(phenyliisocyanate) (MDI) extended with butanediol (BDO), are widely used for implantable cardiac pacing leads. Pacing leads are insulated wires with electrodes that carry stimuli to tissues and biologic signals back to implanted pulse generators. The use of polyether polyurethane elastomers as insulation on such leads has provided significant advantage over silicone rubber, primarily because of the higher tensile strength and elastic modulus of the polyurethanes.

Examples of commercial polyurethanes that can be used in connection with the invention include TECOFLEX®, TECOTHANE®, and BIOSPAN® polyurethanes. TECOFLEX® segmented polyurethanes are a family of aliphatic, polyether-based thermoplastic polyurethanes (TPUs) available over a wide range of durometers, colors, and radiopacifiers. These resins are generally easy to process and typically do not yellow upon aging. TECOTHANE® segmented polyurethanes are a family of aromatic, polyether-based TPUs available over a wide range of durometers, colors, and radiopacifiers. Generally, TECOTHANE® resins exhibit improved solvent resistance and biostability when compared with TECOFLEX® resins of equal durometer. As with any aromatic polyurethane, TECOTHANE® resins can tend to yellow upon aging or when subjected to radiation sterilization. BIOSPAN® segmented polyurethane (SPU) is a biomaterial widely used in clinical ventricular assist devices and artificial heart cases. It is one of the most extensively tested biomaterials on the market. BIOSPAN® is an elastomeric biomaterial exhibiting a superior combination of physical and mechanical properties together with biological compatibility.

Further examples of commercial polyurethanes that can be used in connection with the invention include Sancure 2710® and/or Avalure UR 445® (which are equivalent copolymers of polypropylene glycol, isophorone diisocyanate, and 2,2-dimethylolpropionic acid, having the International Nomenclature Cosmetic Ingredient name “PPG-17/PPG-34/IPDI/DMPA Copolymer”), Sancure 878®, Sancure 815®, Sancure 1301′, Sancure 2715®, Sancure 1828®, Sancure 2026®, Sancure 1818®, Sancure 853®, Sancure 830®, Sancure 825®, Sancure 776®, Sancure 850®, Sancure 12140®, Sancure 12619®, Sancure 835®, Sancure 843®, Sancure 898®, Sancure 899®, Sancure 1511®, Sancure 1514®, Sancure 1517®, Sancure 1591®, Sancure 2255®, Sancure 2260®, Sancure 2310®, Sancure 2725®, and Sancure 12471® (all of which are commercially available from BFGoodrich, Cleveland, Ohio), Bayhydrol DLN (commercially available from Bayer Corp., McMurray, Pa.), Bayhydrol LS-2033 (Bayer Corp.), Bayhydrol 123 (Bayer Corp.), Bayhydrol PU402A (Bayer Corp.), Bayhydrol 110 (Bayer Corp.), Witcobond W-320 (commercially available from Witco Performance Chemicals), Witcobond W-242 (Witco Performance Chemicals), Witcobond W-160 (Witco Performance Chemicals), Witcobond W-612 (Witco Performance Chemicals), Witcobond W-506 (Witco Performance Chemicals), NeoRez R-600 (a polytetramethylene ether urethane extended with isophorone diamine commercially available from Avecia, formerly Avecia Resins), NeoRez R-940 (Avecia Resins), NeoRez R-960 (Avecia Resins), NeoRez R-962 (Avecia Resins), NeoRez R-966 (Avecia Resins), NeoRez R-967 (Avecia Resins), NeoRez R-972 (Avecia Resins), NeoRez R-9409 (Avecia Resins), NeoRez R-9637 (Avecia), NeoRez R-9649 (Avecia Resins), and NeoRez R-9679 (Avecia Resins).

In a further aspect, the polymer fibers are aliphatic polyether polyurethanes. Examples of such aliphatic polyether polyurethanes include Sancure 2710® and/or Avalure UR 445®, Sancure 878®, NeoRez R-600, NeoRez R-966, NeoRez R-967, and Witcobond W-320.

In the segmented polymers of the invention, the soft segments can be any of those typically used in segmented polyurethanes, such as those disclosed in U.S. Pat. No. 4,873,308 (Coury et al.). The soft segments can include ether groups, ester groups, carbonate groups, urea groups, branched hydrocarbon groups, silicone groups, and the like. Such groups are typically noncrystallizing. For example, the soft segments can be based upon noncrystallizing hydrocarbon backbones such as dimer acid derivatives, linked by urethane groups to short and/or medium chain length hydrocarbon moieties. The soft segments can also be derived from siloxane diols such as polydimethyl siloxane diol, polyether diols such as polytetramethylene ether glycols, polyester diols such as polyethylene/polypropylene adipate glycol polyester diol, and polycaprolactone polyester diol, and the like. Such diols can include methyl, phenyl, propyl, etc., substitution and can also include carbonol termination that may include any number of methylene units as desired. To improve the biocompatibility of a segmented polyurethane (SPU), 2-methacryloyloxyethyl phosphorylcholine (MPC) copolymer can be blended in the SPU by a solvent evaporation method from a homogeneous solution containing both SPU and MPC copolymer.

4. Supplementary Materials

In one aspect, the conduit can further comprise a supplementary material. The supplementary material can be any supplementary material known to those of skill in the art and can be selected to modify the properties of the polymer fibers. For example, cellular adhesion can be improved by incorporation of soluble type I collagen into the scaffold by co-spinning the collagen from a solution of 1,1,1,3,3,3-hexafluoro-2-propanol using a dual needle system. The supplementary material can be added to the spinning solution to produce the polymer fibers. In a further aspect, the supplementary material can be added to the polymer fibers after spinning. In various aspects, the supplementary material comprises polymer fibers, a polymer network, or a coating.

In one aspect, the supplementary material comprises collagen, fibrin, chitin, laminin, polyethylene glycol, or a mixture thereof. In a further aspect, the supplementary material comprises a synthetic peptide, a polysaccaride, a proteoglycan, or an extracellular matrix component.

5. Additives

In one aspect, the conduit can further comprise at least one additive. The additive can be any additive known to those of skill in the art and can be selected to modify the properties of the polymer fibers. The additive can be added to the spinning solution to produce the polymer fibers. In a further aspect, the additive can be added to the polymer fibers after spinning.

In one aspect, the additive comprises a pharmaceutically active agent, for example, an antithrombogenic agent such as heparin. In this aspect, once the conduit is implanted into a subject, the additive can then be released from the porous conduit into a subject. In this aspect, the conduit can serve as an additive, for example pharmaceutically active agent, delivery system.

6. Substantially Circumferential Fibers

In one aspect, the polymer fibers are substantially aligned. That is, the fibers are “wrapped” in a substantially aligned spiral to comprise the tubular body. In one aspect, the substantially aligned circumferential fibers of the conduit can be produced using a spinning, for example an electrospinning, technique. A schematic of an electrospinning technique is shown in FIG. 2.

In a further aspect, the polymer fibers are not substantially aligned. That is, the fibers can be, for example, randomly positioned.

Electrospinning is an atomization process of a fluid which exploits the interactions between an electrostatic field and the fluid. That is, electrospinning is a method of electrostatic extrusion used to produce sub-micron sized fibers. In one aspect, the fluid can be a conducting fluid. Also known within the fiber forming industry as electrostatic spinning, the process of electrospinning generally involves the creation of an electrical field at the surface of a liquid. Electrospinning processes are described for example in Fong, H.; Reneker, D. H., J. Polym. Sci., Part B, 37 (1999) at 3488 and in J. Doshi et al., Electrospinning Process and Applications of Electrospun Fibers, Journal of Electrostatics, vol. 35, No. 2, (1995) at 151-160. When an external electrostatic field is applied to a conducting fluid (e.g., a semi-dilute polymer solution or a polymer melt), a suspended conical droplet is formed, whereby the surface tension of the droplet is in equilibrium with the electric field. Electrostatic atomization occurs when the electrostatic field is strong enough to overcome the surface tension of the liquid. The resulting electrical forces create a jet of liquid which carries electrical charge. Thus, the liquid jets may be attracted to other electrically charged objects at a suitable electrical potential. As the jet of liquid elongates and travels, it will harden and dry. Fibrils of nanometer-range diameter can be produced. The hardening and drying of the elongated jet of liquid may be caused by cooling of the liquid, by evaporation of a solvent, or by a curing mechanism. The produced fibers are collected on a suitably located, oppositely charged receiver and subsequently removed from it as needed, or directly applied to an oppositely charged generalized target area.

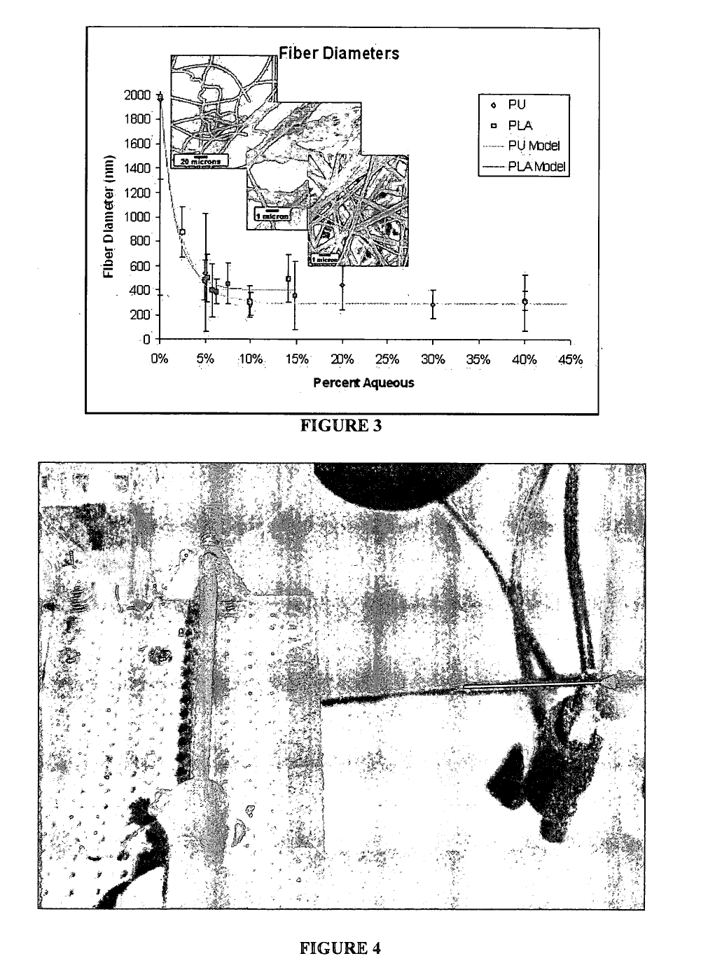

Fibers can be electrospun from high viscosity polymer melts or polymers dissolved in volatile solvents; the end result is a non-woven mesh of fiber. Solution viscosity can be controlled by modifying polymer concentration, molecular weight, and solvents. Electric field properties can be controlled by modifying bias magnitude or tip-to-target distance. Polymers can be co-spun from same the solution and the polymer phase can be selectively removed. Further, fibers can be electrospun from a multiphasic polymer solution or from an emulsion. For example, polyurethane fibers can be electrospun from a multiphasic polyurethane solution. Emulsifying the solution can increase the solution viscosity, thereby inducing fiber formation at lower concentrations. The resultant fibers can be created having diameters as a function of aqueous content. This relationship is shown, for example, in FIG. 3. The curve can be similar to that observed with a poly(lactic acid) system.

In a further aspect, a solution or an emulsion containing a polymer can be electrospun and collected by a receiver. For example, the receiver can be a rapidly rotating axle, spindle, or mandrel, as shown in FIG. 4. Accordingly, the produced fibers can form a tubular structure, that is, the fibers can be aligned circumferentially. A combination of controlling the electric field and high receiver pickup rate can be used to align electrospun polymer fibers circumferentially over long distances (>4 cm). Such alignment can facilitate circumferential alignment of smooth muscle cells (SMC) across the exterior surface of the conduit when used as a vascular prosthesis. More specifically, controlled SMC alignment can be achieved by using mechanical forces and matrix orientation. In one aspect, long-range alignment of fibers parallel to the short axis of the conduit can promote circumferential orientation of SMC when the conduit is used as a vascular graft. Previous studies have shown that for fiber diameters smaller than cell diameters, cells align along the direction of the fiber. Accordingly, SMC can align in the direction of cyclic strain. In the present conduit, SMC can align perpendicular to fluid flow. That is, the SMC can align substantially perpendicular to the length of the lumen.

In one aspect, fiber diameter can be an important parameter for controlling cell morphology and differentiation and proliferation. Polymer fibers, or fibrils, of nanometer-range diameter can be produced. By “nanometer-range diameter,” it is meant to include fibrils ranging in diameter from approximately 1 nanometer to approximately 10,000 nanometers (nm). For example, the fibrils can range in diameter from about 1 nm to about 10,000 nm, from about 10 nm to about 1,000 nm, about 50 m to about 500 nm, about 100 nm to about 500 nm, about 100 nm to about 500 nm, about 100 nm to about 500 nm, about 100 nm to about 500 nm, about 500 nm to about 1,000 nm, about 100 nm to about 200 nm, about 200 nm to about 300 nm, about 300 nm to about 400 nm, about 400 nm to about 500 nm, or about 500 nm to about 600 nm. In a further aspect, the polymeric fibers can, for example, have diameters of less than about 2 μm. For example, polymeric fibers can have diameters of less than about 1 μm, or even less than about 0.5 μm.

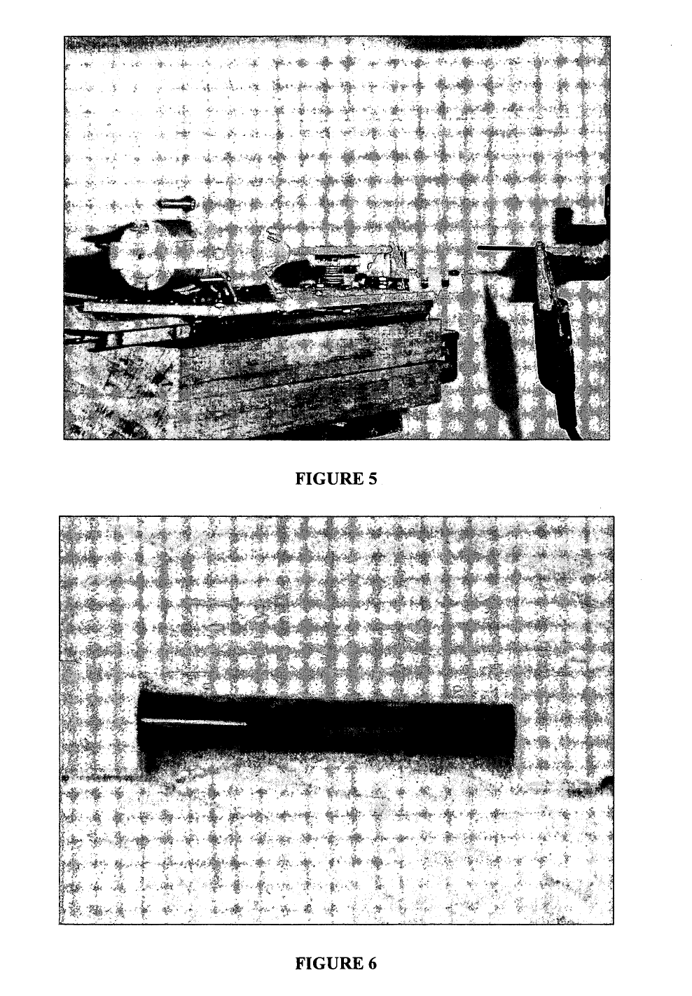

Electrospinning nanometer-range diameter polymer fibrils in conjunction with a rotating mandrel (e.g., about 4 mm diameter) target (FIG. 5) can be used to engineer conduits that possesses circumferential fiber alignment throughout the length of the scaffold (3-4 cm). By using an emulsion system, fibers can be spun out of lower solution concentration under nominal conditions by exploiting the shear-thinning behavior of emulsions. In addition, the emulsion system provides a unique way to tailor the dielectrics and conductivity of the polymer solution, by the addition of salts and other organic electrolytes in the aqueous phase. By controlling the electrical properties of polymer solution, the formation of the Taylor cone can be controlled more precisely to minimize jet whipping and hence fiber alignment.

7. Microscale Features

In one aspect, the conduit can be provided having microscale features disposed at the interior surface. That is, the interior surface can comprise microscale features.

In one aspect, the microscale features can be randomly disposed at the interior surface. In a further aspect, the microscale features can be disposed in an ordered arrangement at the interior surface. In a further aspect, the arrangement of the microscale features can be predetermined. In an even further aspect, the microscale features can be patterned. That is, the microscale features can be provided by transfer from a pattern. For example, the microscale features can be provided by physical transfer from the complementary features of a pattern. By complementary, it is meant that the microscale features of the interior surface are a negative relief of the microscale features of the pattern. In this aspect, a pattern can have microscale features, and the conduit is prepared in contact with the pattern. During preparation, the interior surface of the conduit is provided having microscale features as a negative relief of the microscale features of the pattern. In one aspect, the rotating mandrel used to collect electrospun polymer fibers during preparation of the conduit if the invention can function as a pattern. In this aspect, before preparation of the conduit, the mandrel is adapted to include complementary microscale features.

By “microscale,” it is meant to include features having dimensions ranging from approximately 1 micrometer to approximately 1,000 micrometers. In one aspect, the microscale features have dimensions of from about 1 μm to about 1,000 μm, for example, from about 10 μm to about 1,000 μm, from about 20 μm to about 120 μm, from about 40 μm to about 80 μm, from about 50 μm to about 60 μm, from about 50 μm to about 100 μm, from about 100 μm to about 1,000 μm, from about 1 μm to about 500 μm, from about 10 μm to about 500 μm, from about 500 μm to about 1,000 μm, from about 50 μm to about 500 μm, from about 100 μm to about 200 μm, or from about 200 μm to about 300 μm.

In one aspect, the microscale features are reservoirs. By “reservoirs,” it is meant that the microscale features comprise a surface having substantially irregular-shaped voids therein. The reservoirs can have an average width of from about 10 μm to about 500 μm, for example, from about 10 μm to about 100 μm, from about 10 μm to about 200 μm, from about 10 μm to about 300 μm, from about 10 μm to about 400 μm, from about 50 μm to about 100 μm, from about 50 μm to about 100 μm, from about 50 μm to about 500 μm, from about 100 μm to about 200 μm, or from about 200 μm to about 300 μm. The reservoirs can have an average depth of from about 10 μm to about 100 μm, for example, from about 20 μm to about 60 μm, from about 20 μm to about 80 μm, from about 30 μm to about 70 μm, from about 40 μm to about 60 μm, or about 50 μm. In a further aspect, more than one reservoir can be disposed at the interior surface. In a further aspect, a multiplicity of reservoirs is disposed at the interior surface. In a further aspect, reservoirs can be absent from the invention.

In a further aspect, the microscale features can be protrusions or wells. It is understood that both protrusions and wells can be present at the interior surface. In one aspect, the protrusions comprise raised portions extending outward from the interior surface. In a further aspect, the protrusions can be substantially round or substantially square masses extending outward from the interior surface. The protrusions can have an average width of from about 10 μm to about 500 μm, for example, from about 10 μm to about 100 μm, from about 10 μm to about 200 μm, from about 10 μm to about 300 μm, from about 10 μm to about 400 μm, from about 50 μm to about 100 μm, from about 50 μm to about 100 μm, from about 50 μm to about 500 μm, from about 100 μm to about 200 μm, or from about 200 μm to about 300 μm. The protrusions can have an average height of from about 10 μm to about 100 μm, for example, from about 20 μm to about 60 μm, from about 20 μm to about 80 μm, from about 30 μm to about 70 μm, from about 40 μm to about 60 μm, or about 50 μm.

In this aspect, wells comprise substantially round or substantially square voids extending inward from the interior surface. The wells can have an average width of from about 10 μm to about 500 μm, for example, from about 10 μm to about 100 μm, from about 10 μm to about 200 μm, from about 10 μm to about 300 μm, from about 10 μm to about 400 μm, from about 50 μm to about 100 μm, from about 50 μm to about 100 μm, from about 50 μm to about 500 μm, from about 100 μm to about 200 μm, or from about 200 μm to about 300 μm. The wells can have an average depth of from about 10 μm to about 100 μm, for example, from about 20 μm to about 60 μm, from about 20 μm to about 80 μm, from about 30 μm to about 70 μm, from about 40 μm to about 60 μm, or about 50 μm. In a further aspect, more than one protrusion or well can be disposed at the interior surface. In a further aspect, a multiplicity of protrusions and/or wells is disposed at the interior surface. In a further aspect, protrusions and/or wells can be absent from the invention.

In one aspect, the microscale features comprise ridges. In this aspect, a ridge comprises a raised strip extending outward from the interior surface. In one aspect, a ridge can be disposed substantially parallel to the direction of the lumen. In this aspect, the ridge extends along at least a portion of the length of the lumen. In a further aspect, a ridge can be disposed substantially perpendicular to the direction of the lumen. In this aspect, the ridge extends along at least a portion of the circumference of the lumen. The ridges can have an average width of from about 10 μm to about 500 μm, for example, from about 10 μm to about 100 μm, from about 10 μm to about 200 μm, from about 10 μm to about 300 μm, from about 10 μm to about 400 μm, from about 50 μm to about 100 μm, from about 50 μm to about 100 μm, from about 50 μm to about 500 μm, from about 100 μm to about 200 μm, or from about 200 μm to about 300 μm. The ridges can have an average height of from about 10 μm to about 100 μm, for example, from about 20 μm to about 60 μm, from about 20 μm to about 80 μm, from about 30 μm to about 70 μm, from about 40 μm to about 60 μm, or about 50 μm. In a further aspect, more than one ridge can be disposed at the interior surface. In a further aspect, a multiplicity of ridges is disposed at the interior surface. In a further aspect, ridges can be absent from the invention.

In one aspect, the microscale features comprise grooves. In this aspect, a groove comprises a canal or ditch extending inward from the interior surface. In one aspect, a groove can be disposed substantially parallel to the direction of the lumen. In this aspect, the groove extends along at least a portion of the length of the lumen. In a further aspect, a groove can be disposed substantially perpendicular to the direction of the lumen. In this aspect, the groove extends along at least a portion of the circumference of the lumen. The grooves can have an average width of from about 10 μm to about 500 μm, for example, from about 10 μm to about 100 μm, from about 10 μm to about 200 μm, from about 10 μm to about 300 μm, from about 10 μm to about 400 μm, from about 50 μm to about 100 μm, from about 50 μm to about 100 μm, from about 50 μm to about 500 μm, from about 100 μm to about 200 μm, or from about 200 μm to about 300 μm. The grooves can have an average height of from about 10 μm to about 100 μm, for example, from about 20 μm to about 60 μm, from about 20 μm to about 80 μm, from about 30 μm to about 70 μm, from about 40 μm to about 60 μm, or about 50 μm. In a further aspect, more than one groove can be disposed at the interior surface. In a further aspect, a multiplicity of grooves is disposed at the interior surface. In a further aspect, grooves can be absent from the invention.

It is also understood that a combination of reservoirs, protrusions, wells, ridges, and/or grooves can be disposed at the interior surface.

In one aspect, the microscale features comprise grooves disposed at the interior surface of the conduit substantially parallel to the direction of the lumen. In this aspect, the arrangement of the microscale features can be ordered and predetermined. In a further aspect, the ordered and predetermined microscale features are patterned. In a further aspect, the substantially parallel grooves extend along at least a portion of the length of the lumen. The grooves can have, for example, an average width of from about 50 nm to about 100 nm and an average depth of from about 50 nm to about 60 nm.

In a further aspect, the conduit can be a tubular prosthesis having reservoirs with a width of less than about 100 μm; for example, the reservoirs can have a width of less than about 50 μm. In a further aspect, a prosthesis can have reservoirs spaced approximately 100 μm apart. The reservoirs can be, for example, spaced approximately 50 μm apart. More specifically, the reservoirs can be spaced, for example, 40, 30, 20, or even 10 μm apart. Further, the reservoirs can be regularly or irregularly spaced.

8. Cells Adhered to the Conduit

Cell cultures can be established from mammalian, or other, tissue sources by dissociating the tissue or by an explant method. Primary cultures can be established and cryopreserved in master cell banks from which portions of the bank can be thawed, seeded, and subcultured to expand cell numbers. To populate an acellular scaffold with cells, the scaffold can be placed in a culture dish or flask and contacted by immersion in media containing suspended cells.

Although human cells are preferred for use in the invention, the cells to be used are not limited to cells from human sources. Cells from other mammalian species including, but not limited to, equine, canine, porcine, bovine, ovine, and murine sources may be used. Cell donors may vary in development and age. Embryonic progenitor cells such as stem cells may be used in the invention and induced to differentiate to develop into the desired tissue. In addition, genetically engineered cells that are spontaneously, chemically, or virally transfected may also be used in this invention. For those aspects that incorporate more than one cell type, mixtures of normal and genetically modified or transfected cells may be used, and mixtures of cells of two or more species or tissue sources may be used, or both.

Recombinant or genetically-engineered cells may be used to create a tissue construct that acts as a drug delivery graft for a subject needing increased levels of natural cells products or treatment with a therapeutic. The cells can produce and deliver to the patient via the graft recombinant cells products, growth factors, hormones, peptides or proteins for a continuous amount of time or as needed when biologically, chemically, or thermally signaled due to the conditions present in the patient.

A tubular prosthesis can have smooth muscle cells in contact with the exterior surface of the tubular structure. Smooth muscle cells can form a confluent tubular sheet surrounding the tubular structure. A tubular prosthesis can have endothelial cells in contact with the interior surface of the tubular structure. Endothelial cells can be positioned within linear reservoirs in the interior surface or can form a confluent tubular sheet in contact with the interior surface of the tubular structure.

In a further aspect, the conduit can further comprise at least one cell adhered to the exterior surface and at least one cell adhered to the interior surface. The at least one cell adhered to the exterior surface and the at least one cell adhered to the interior surface can be the same cell type or can be different cell types.

Cells that can be used in connection with the invention include a chondroblast, a chondrocyte, a fibroblast, a transfected fibroblast, an endothelial cell, an osteoblast, an osteocyte, an epithelial cell, an epidermal cell, a mesenchymal cell, a hemopoietic cell, an embryoid body, a nerve cell, a Schwann cell, a glial cell, a stem cell, dorsal root ganglia, and mixtures thereof.

a. Exterior Surface

In one aspect, the conduit can comprise at least one cell adhered to the exterior surface. In a further aspect, the at least one cell comprises a smooth muscle cell or a transfected fibroblast. In a further aspect, the at least one cell comprises a fibroblast transfected to release nerve growth factor. In one aspect, more than one cell type can be used in connection with the exterior surface. In a further aspect, cells can be substantially absent from the exterior surface.

In an even further aspect, the conduit can comprise a multiplicity of cells in contact with the exterior surface. In a still further aspect, the cells comprise a confluent tubular sheet in contact with the exterior surface.

b. Interior Surface

In one aspect, the conduit can comprise at least one cell adhered to the interior surface. In a further aspect, the at least one cell comprises an endothelial cell. In a further aspect, the at least one cell comprises at least one nerve cell, Schwann cell, glial cell, stem cell, or a mixture thereof. In one aspect, a multiplicity of endothelial cells can be in contact with the interior surface. In a further aspect, cells can comprise a confluent tubular sheet in contact with the interior surface.

In one aspect, more than one cell type can be used in connection with the interior surface. In a further aspect, cells can be substantially absent from the interior surface.

9. Multilayered Conduits

In a further aspect, the conduits of the invention can comprise multilayered conduits. That is, the tubular body of the conduit can be formed from more than one layer. In one aspect, one or more inner layer(s) of, for example poly(lactide-co-glycolide), can be spun or electrospun onto a rotating mandrel. The inner layer(s) can optionally comprise substantially circumferential fibers and/or can comprise highly aligned fibers. In one aspect, the inner layer(s) can have microscale features disposed at the interior surface. In a further aspect, the microscale features can be complementary to microscale features present on the mandrel. In a further aspect, one or more further layer(s) of, for example segmented polyurethane, can be then spun or electrospun onto the rotating mandrel and the inner layer(s). The further layer(s) can optionally comprise substantially circumferential fibers and/or can comprise highly aligned fibers. In a further aspect, one or more outer layer(s) of, for example poly(lactic acid), can be then spun or electrospun onto the rotating mandrel and the further layer(s). The outer layer(s) can optionally comprise substantially circumferential fibers and/or can comprise highly aligned fibers.

In one aspect, the substantially tubular body comprises at least one first layer of substantially circumferential electrospun polymer fibers and at least one second layer of polymer fibers, wherein the first layer is different from the second layer. In a further aspect, the at least one second layer of polymer fibers is disposed inside the at least one first layer of substantially circumferential electrospun polymer fibers. That is, the second layer can be disposed closer to the lumen than the first layer. In a further aspect, the at least one first layer of substantially circumferential electrospun polymer fibers is disposed inside the at least one second layer of polymer fibers. That is, the second layer can be disposed further from the lumen than the first layer.

In one aspect, the at least one second layer of polymer fibers comprises nonbiodegradable polymer fibers. In a further aspect, the polymer fibers can be biodegradable. In a further aspect, the at least one second layer of polymer fibers comprises substantially circumferential electrospun polymer fibers. The polymer fibers can comprise any polymer known to those of skill in the art, for example, the polymers disclosed herein. In particular, the at least one second layer of polymer fibers can be poly(lactic acid), poly(glycolic acid), poly(lactide-co-glycolide), or a mixture thereof.

In a further aspect, the conduit can further comprise at least one third layer of polymer fibers, wherein the at least one first layer of substantially circumferential electrospun polymer fibers is disposed between the at least one second layer of polymer fibers and the at least one third layer of polymer fibers.

In one aspect, the substantially tubular body comprises at least one first layer of substantially circumferential polymer fibers and at least one second layer of polymer fibers, wherein the first layer is different from the second layer. In a further aspect, the at least one second layer of polymer fibers is disposed inside the at least one first layer of substantially circumferential polymer fibers. That is, the second layer can be disposed closer to the lumen than the first layer. In a further aspect, the at least one first layer of substantially circumferential polymer fibers is disposed inside the at least one second layer of polymer fibers. That is, the second layer can be disposed further from the lumen than the first layer.

In one aspect, the at least one second layer of polymer fibers comprises nonbiodegradable polymer fibers. In a further aspect, the polymer fibers can be biodegradable. In a further aspect, the at least one second layer of polymer fibers comprises substantially circumferential electrospun polymer fibers. The polymer fibers can comprise any polymer known to those of skill in the art, for example, the polymers disclosed herein. In particular, the at least one second layer of polymer fibers can be poly(lactic acid), poly(glycolic acid), poly(lactide-co-glycolide), or a mixture thereof.

In a further aspect, the conduit can further comprise at least one third layer of polymer fibers, wherein the at least one first layer of substantially circumferential polymer fibers is disposed between the at least one second layer of polymer fibers and the at least one third layer of polymer fibers.

It is understood that the first layer(s) of substantially circumferential polymer fibers, the first layer(s) of substantially circumferential electrospun polymer fibers, the second layer(s) of polymer fibers, the third layer of polymer fibers can be used in connection with the supplementary materials, the additives, the methods of making, the methods of using, and the applications of the invention. It is also understood that the second layer(s) of polymer fibers and/or the third layer of polymer fibers can be absent from the invention.

10. Vascular Prostheses

In one aspect, the conduit can comprise a vascular prosthesis. In this aspect, the vascular prosthesis can comprise a substantially tubular body comprising substantially aligned, substantially circumferential, electrospun polyurethane fibers; wherein the body has an exterior surface, an interior surface, and a lumen with a diameter of from about 2 mm to about 4 mm extending therethrough; wherein the body has microscale grooves disposed at the interior surface substantially parallel to the lumen; wherein the grooves have an average width of from about 50 μm to about 100 μm and an average depth of from about 20 μm to about 60 μm; wherein at least one smooth muscle cell is adhered to the exterior surface; and wherein at least one endothelial cell is adhered to the interior surface.

In a further aspect, the vascular prosthesis, also referred to as a scaffold, comprises a tubular structure with an interior surface and an exterior surface. The interior surface can be modified such that endothelial or epithelial cells of any type can attach thereto. For example, the interior surface can be porous allowing for endothelial cells to attach and grow into or line the interior surface of the device. The porosity can be varied to increase or decrease the rate or amount of cellular attachment, or can be varied based on the desired application or location of the scaffold within the subject. The exterior surface can be similarly modified to allow cellular in-growth or coating by a desired cell type. For example, the exterior surface can be modified to allow or promote in-growth or coating by smooth muscle cells. The cell types that can attach to, and grow into or cover, the scaffold, however, are not intended to be limited to endothelial cells and smooth muscle cells on the interior or exterior surfaces, respectively. Any cell type that lines the interior or coats the exterior of a tubular structure within a subject, human or animal, can be used. For example, transitional epithelial cells, can be attach to the interior surface of the scaffold when the scaffold is used within the urinary system. Thus, epithelial cells of any type can be attached to the interior of the scaffold. Similarly, any other desired cell types can attach to exterior surface of the scaffold. For example, such cells include, but are not limited, to cardiac muscle cells, chondrocytes, and fibrocytes. One of skill in the art can determine what type of cell to attach to the interior or exterior surface of the scaffold based on the desired location of the prosthesis. Moreover, different cells will attach to the scaffold in vivo depending on where in the body the scaffold is placed.

Alternatively, the prosthesis or scaffold can be used, for example, to replace a length of tubular organs such as vasculature, ureters, urethra, esophagus, trachea, intestine, vas deferens and fallopian tubes. These organs have a basic tubular shape with an outer surface and an inner luminal surface. The scaffold's inner and exterior surfaces can be lined or coated with cells in vitro or in vivo. For example, if the scaffold is to be used to replace a section of vasculature, endothelial cells can be cultured and attached onto the scaffold in vitro before placement into a subject. Similarly, if a section of urethra or ureter is to be replaced, the appropriate epithelial cell type can be cultured and attached to the scaffold in vitro prior to insertion into the ureter or urethra. Alternatively, the scaffold can be inserted into the appropriate region of the body without any cellular attachment. If the scaffold is inserted without cells, the subjects own cells attach to the scaffold in vivo. Moreover, a combination of the in vitro and in vivo approach can also be used. For example, a given number of cells can be attached or seeded onto the scaffold in vitro, and then the scaffold can be inserted into a subject where additional cells of the subject can attach to the already seeded prosthesis.

11. Stents

In one aspect, the conduit can comprise a stent. Stents are known in the art and are commonly used as a tubular structure left inside the lumen of a duct to relieve an obstruction. Commonly, stents are inserted into the lumen in a non-expanded form and are then expanded autonomously (or with the aid of a second device) in situ. A typical method of expansion occurs through the use of a catheter mounted angioplasty balloon, which is inflated within the stenosed vessel or body passageway, in order to shear and disrupt the obstructions associated with the wall components of the vessel and to obtain an enlarged lumen.

In the absence of a stent, restenosis may occur as a result of elastic recoil of the stenotic lesion. Although a number of stent designs have been reported, these designs have suffered from a number of limitations. These include restrictions on the dimension of the stent.

Other stents are described as longitudinally flexible but consist of a plurality of cylindrical elements connected together. This design has at least one important disadvantage, for example, according to this design, protruding edges occur when the stent is flexed around a curve raising the possibility of inadvertent retention of the stent on plaque deposited on arterial walls. This can cause a stent to form emboli or move out of position and further cause damage to the interior lining of healthy vessels.

In one aspect, the conduit if the present invention comprises aligned circumferential polymer fibers and is, therefore, both longitudinally rigid and radially compliant. Accordingly, in one aspect, the conduit can avoid inadvertent retention of the stent on plaque deposited on arterial walls. Furthermore, in a further aspect, the stent of the present invention can further comprise cells, for example, endothelial cells adhered at the interior surface. In a further aspect, stent of the present invention can further comprise one or more of the additives, for example a pharmaceutically active agent, such as rapamycin, cladribine, heparin, nitrous oxide, or any other known drugs, either alone or in combination.

12. Nerve Regeneration Scaffolds

In one aspect, the conduit can comprise a nerve regeneration scaffold. That is, the conduit can provide tissue regenerational supportive material.

In this aspect, the nerve regeneration scaffold comprises a substantially tubular body comprising substantially aligned, substantially circumferential, electrospun polyurethane fibers; wherein the body has an exterior surface, an interior surface, and a lumen with a diameter of from about 2 mm to about 4 mm extending therethrough; wherein the body has microscale ridges or grooves disposed at the interior surface substantially parallel to the lumen; and wherein at least one transfected fibroblast cell is adhered to the exterior surface.

In a further aspect, the nerve regeneration scaffold of claim, further comprises at least one nerve cell, Schwann cell, glial cell, stem cell, or a mixture thereof adhered to the interior surface.

In addition to vascular grafts and stents, the present invention can be applied to other tissue engineering projects, for example, electrospun nerve guidance channels for peripheral and central nerve regeneration. These constructs are prepared using a combination of extracellular matrix (ECM) proteins and synthetic, non-degradable polymers to leverage the body's natural nerve regeneration process. Specifically, conduits consisting mostly of a non-degradable polymer can be produced with ECM proteins such as collagen and laminin disposed at the interior surface of the lumen of the conduit to promote axonal outgrowth and nerve cell attachment. Genetically modified fibroblasts, transfected to produce nerve growth factor (NGF) or any other combination of growth factors, can be seeded on the outside of the graft. The electronspun biohybrid conduits of the invention afford several advantages over conventional techniques. First, the porous conduit allows for the diffusion of bioactive molecules, for example NGF secreted by fibroblasts, into the nerve injury site. While transfected cells typically only demonstrate temporal expression of genes, the present invention can leverage this fact, since enhanced NGF concentrations are only necessary during the healing period of the nerve and not for the full lifetime of the conduit. Second, the non-degradable component of the conduit isolates the site of the injury, thereby providing a physical barrier against fibroblast infiltration and possible scarring of the nerve bundle.

### D. METHODS OF MAKING SYNTHETIC CONDUITS

In one aspect, the invention relates to a method of preparing a synthetic conduit comprising the step of electrospinning a solution of polymer onto a rotating mandrel bearing microscale features, thereby providing a substantially tubular body comprising substantially circumferential electrospun polymer fibers; wherein the body has an exterior surface, an interior surface, and a lumen extending therethrough; and wherein the body has complementary microscale features disposed at the interior surface. In this aspect, the method can further comprise the step of removing the body from the mandrel.

In a further aspect, the invention relates to a method of preparing a synthetic conduit comprising the step of spinning a polymer onto a rotating mandrel bearing microscale features, thereby providing a substantially tubular body comprising substantially circumferential electrospun polymer fibers; wherein the body has an exterior surface, an interior surface, and a lumen extending therethrough; and wherein the body has complementary microscale features disposed at the interior surface. In this aspect, the method can further comprise the step of removing the body from the mandrel.

In one aspect, the synthetic conduit can be a vascular prosthesis. In this aspect, the method of preparing a vascular prosthesis comprises the steps of electrospinning a solution of polymer onto a rotating mandrel bearing microscale features, thereby providing a substantially tubular body comprising substantially circumferential electrospun polymer fibers; wherein the body has an exterior surface, an interior surface, and a lumen extending therethrough; and wherein the body has complementary microscale features disposed at the interior surface; adhering at least one smooth muscle cell to the exterior surface; and adhering at least one endothelial cell to the interior surface. Then, the body is removed from the mandrel.

In a further aspect, the synthetic conduit can be a nerve regeneration scaffold. In this aspect, the method of preparing a nerve regeneration scaffold comprises the steps of electrospinning a solution of polymer onto a rotating mandrel bearing microscale features, thereby providing a substantially tubular body comprising substantially circumferential electrospun polymer fibers; wherein the body has an exterior surface, an interior surface, and a lumen extending therethrough; and wherein the body has complementary microscale features disposed at the interior surface; and adhering at least one transfected fibroblast to the exterior surface. In this aspect, the method can further comprise the step of adhering at least one nerve cell, Schwann cell, glial cell, stem cell, or a mixture thereof to the interior surface. Then, the body is removed from the mandrel.

1. Electrospinning

The technique of electrospinning, also known within the fiber forming industry as electrostatic spinning, of liquids and/or solutions capable of forming fibers, is well known and has been described in a number of patents as well as in the general literature.

Typically, the process of electrospinning generally involves the creation of an electrical field at the surface of a liquid. Fibers produced by this process have been used in a wide variety of applications, and are known, from U.S. Pat. Nos. 4,043,331 and 4,878,908, to be particularly useful in forming non-woven structures. The resulting electrical forces create a jet of liquid which carries electrical charge. Thus, the liquid jets maybe attracted to other electrically charged objects at a suitable electrical potential. As the jet of liquid elongates and travels, it will harden and dry. The hardening and drying of the elongated jet of liquid may be caused by cooling of the liquid, i.e., where the liquid is normally a solid at room temperature; evaporation of a solvent, e.g., by dehydration, (physically induced hardening); or by a curing mechanism (chemically induced hardening). The produced fibers are collected on a suitably located, oppositely charged receiver and subsequently removed from it as needed, or directly applied to an oppositely charged generalized target area.

In one aspect, electrospinning (ES) is an atomization process of fluid which exploits the interactions between an electrostatic field and the fluid. In one aspect, the fluid can be a conducting fluid. During electrospinning, fibers with micron or sub-micron sized diameters are extruded be means of an electrostatic potential from a polymer solution (see U.S. Pat. No. 1,975,504 to Formhals). When an external electrostatic field is applied to a fluid (e.g., a semi-dilute polymer solution or a polymer melt), a suspended conical droplet is in equilibrium with the electric field. Electrostatic atomization occurs when the electrostatic field is strong enough to overcome the surface tension of the liquid. The liquid droplet then becomes unstable and a tiny jet is ejected from the surface of the droplet. As it reaches a grounded target, the material can be collected as an interconnected web containing relatively fine, i.e. small diameter, fibers. The resulting films (or membranes) from these small diameter fibers have very large surface area to volume ratios and small pore sizes. This process typically yields non-woven mats or felts composed of round fibers that are extremely pliable. Due to their high-surface area and good mechanical characteristics, electrospun meshes have traditionally found applications in filtration and composite reinforcement. For the very same reasons, felts and meshes derived from biocompatible polymers such as poly(lactic acid) and its copolymer with glycolic acid and other polyesters are being explored as substrates (scaffolds) for association of cells in the engineering of tissue (see Kenawy et al., Biomaterials, 2003, 24 (6), 907 describing making a fiber by electrospinning process from a single-phase system containing ethylene vinyl alcohol, 70% propanol and 30% water). Such pliable porous media is particularly suited for engineering of skin, vascular, and neural prostheses.

Electrospun materials possess a high aspect ratio to allow for cell attachment and spreading, which is a desired property for tissue engineering (TE) applications. The longest axis of a spread cell is typically around about 5 to about 10 micrometers. The ES process is typically not amenable to significant modifications. Parameters that can be varied in the ES process are the electric field, the distance between the “Taylor Cone” and the target, and the polymer solution viscosity (Fridrikh et al., G. C. Phys Rev Lett. 2003, 90(14), 144502). Due to the complexity of the fiber forming process, very few attempts have been made to alter geometry of electrospun fibers. Recently, Reneker and coworkers have observed the formation of branched and ribbon-like fibers in some solvent systems and have attributed this to the collapse of a polymer skin due to buckling instability similar to that seen in garden hoses (see Koombhongse et al., Polym. Sci.: Part B: Polym. Phys. 2001, 39, 2598-2606). However, the formation of such fibers is not achievable in a predictable manner under generally known ES operating conditions. U.S. Pat. Nos. 4,323,525 and 4,689,186 to Bornat, incorporated by reference herein, are directed to processes for the production of tubular products by electrostatically spinning a liquid containing a fiber-forming material.

Toward this objective, the present invention, in one aspect, can use electrospinning (ES) to produce the tubular conduits (also referred to herein as “scaffolds”). ES is a process through which fibers with micron or sub-micron sized diameters are extruded from a polymer solution by means of an electrostatic potential (FIG. 2). In a typical ES process, the polymer solution is injected through a nozzle while being subjected to a high voltage DC field (e.g., 5-30 kV). Under such conditions, the polymer solution erupts into a “Taylor Cone” due to the droplet being subjected to a phenomenon called “Raleigh's Instability,” which leads to whipping of the polymer jet. As the jet is propelled, the formation of fibers is facilitated by solvent evaporation and thinning of the jet. The parameters that can be varied to affect fiber morphology include the electric field strength, the distance between the “Taylor Cone” and the target and viscosity of polymer solution. Due to the complexity of the “Taylor Cone” formation, most attempts at controlling fiber morphology have focused on controlling polymer solution properties. This can be achieved by either increasing the polymer concentration or molecular weight or increasing volatility of the organic solvent; all of which accelerate the rate at which the polymer fibers solidifies during spinning. In general, increasing viscosity and solvent volatility results in thicker fibers.

A limitation of conventional approaches is that they do not enable altering of other fiber properties such as aspect ratio (rounder versus flatter fibers) and fiber porosity both of which can severely impact cellular interactions by increasing surface area, which can be a desired property in cell contacting applications and tissue engineering (TE). In contrast, the present invention demonstrates that by using an unstable water/oil emulsion system (i.e., a polymer solution in an organic solvent emulsified with an aqueous phase), the shear thinning behavior of emulsions can be leveraged to spin fibers from polymer solution at low concentration which under normal conditions are not suitable for electrospinning. Using this approach, polymers such as PU and poly(L-lactic acid) have been spun into fibrous mats with fiber diameters ranging from about 10 nm to about 1,000 nm, for example, from about 300 nm to about 2 μm (FIG. 3). Furthermore, in this emulsion-based system, the less volatile water phase has a templating effect on the polymer fiber formation, enabling control over fiber morphology as well. Depending on the modulus of the polymer, fibers ranging from cylindrical, porous to flat-ribbon like can be obtained (FIG. 3).

Currently, the diameters of electrospun fibers are achieved by changing polymer concentration or solvent systems. Here, we present a simple and general approach to controlling fiber diameter without altering the polymer concentration or solvent system. By using an unstable emulsion (water in oil, with small amounts of polymer surfactant), we are able to predictably vary the rheological properties of the multiphase solution, thereby controlling the final polymer fiber dimensions. In addition, using a multiphase solution allows for a templating effect, giving us control over fiber porosity and shape. We have demonstrated that this multiphase approach works in three polymer/solvent systems. In our PLA/chloroform system, we have electrospun fibers possessing sub-micron diameters (around 400 nm) with ribbon-like and porous morphology at a polymer concentration (2% w/w) that typically yields round fibers with fiber diameters 5-times greater (2 microns). An over two-fold decrease in fiber diameter can be achieved with the addition of just 5% aqueous phase emulsified into the polymer/organic solvent solution. Using emulsions to control fiber shape, diameter, and porosity has desirous applications in many fields including scaffold engineering for vascular, renal, and neural regeneration.

Good mechanical properties, high surface area to weight ratios, and pliability have made electrospun fibers candidates for a wide range of applications in filtration and composite reinforcement. These characteristics, combined with specific polymer properties, also make electrospun felts ideal for tissue engineering scaffolds as well as drug delivery devices.

Fiber diameter is typically controlled by changing electric field strength (either by changing applied voltage or tip-to-target distance), changing evaporation rates (via changing the spinning environment or using solvents of different volatilities), or by changing polymer concentration. The last method enjoys particular popularity among researchers since polymer concentration is an easy variable to control and can have repeatable and drastic effects on fiber diameters. This method works by changing both the amount of solvent that must evaporate before a solid fiber precipitates from the solution and by changing the viscosity of the solution, and hence, “Taylor cone” formation and final jet diameter.

In conventional methods, surface geometry and morphology of electrospun nanofibers has been more difficult to modify. Typical electrospun fibers adopt a circular cross-section, though porous and flat fiber morphologies have been observed in several polymer/solvent systems, but little research has found success at controlling these morphologies. Common techniques used to modify fiber cross-sectional shape have been to cospin polymers and selectively remove certain polymer phases. More recent approaches have succeeded in producing hollow fiber morphologies by using an immiscible second phase and coaxial spinnerets. Both techniques involve either complicated processing steps or specialized electrospinning apparatus to achieve the desired final shape.

In contrast, the present invention employs a new technique to modulate both fiber morphology and diameter. By emulsifying a second phase into the polymer/volatile solvent solution, the fiber diameter can be decreased by an order of magnitude using a single polymer concentration (in, e.g., the organic phase of the emulsion). Additionally, a range of fiber morphologies ranging from common circular cross-sections, to varying amounts of porosity, to flat, ribbon-like polymer fibers, has been observed with the techniques of the invention. Producing these fibers using the present inventive technique can require neither additional processing steps to selective remove components of the fiber, nor complicated modifications to the traditional electrospinning setup.

One of the most modulated parameters in conventional electrospinning techniques involves changing the concentration of the dissolved polymer. This typically has the effect of being able to control the final fiber diameter by changing how the fiber formation process and timescale during electrospinning. One of the more important parameters coupled with polymer concentration is the viscosity of the solution. Viscosity plays a large role in “Taylor cone” formation and stability.

However, changing the concentration of the polymer solution has two limits. Low concentration solutions can lack the viscosity to properly form a “Taylor cone.” In conventional techniques, instead of drawing a single, electrified jet from the spinneret, the jet is broken down into multiple droplets. This process is called electrospraying and has been utilized in processes like applying surface coatings and inkjet printing. However, the electrospraying process lacks fiber forming properties and results in either a coating of connected droplets or a smooth coating of the dissolved polymer.

At high polymer concentration limits, there are the practical limits of being able to handle such a viscous fluid/gel and feed it to the spinneret. Polymer solutions that are too concentrated are difficult to manipulate and tend to clog the electrospinning apparatus. In addition, extremely high field strengths are required to overcome surface tension to properly form the “Taylor cone.” Such high voltages can be impractical to produce or dangerous. As a result, for practical purposes, most fibers produced by conventional techniques at high polymer concentrations tend to have large diameters that are more easily produced using commercially available techniques.

In one aspect, by adding a second phase to the solution, the methods of the invention artificially increase the viscosity of the spinning solution allowing for the formation of a “Taylor cone” at polymer concentrations that typical electrospray. While not wishing to be bound by theory, the mechanism behind this increase in viscosity is widely believed to be the same mechanism observed in everyday culinary ingredients such as whipped cream and mayonnaise. That is, an increased interaction between the multiple phases can create a higher viscosity than the component parts individually. Multicomponent systems comprising the solvents and polymers of the invention, for example, a polyurethane/chloroform:THF (1:1) system, a poly(1-lactic acid)/chloroform:NMP system, or a poly(ethylene co vinyl acetate)/methylene chloride:NMP system, can be used to provide increased interaction between multiple phases, thereby creating a higher viscosity for the system. In this aspect, it can be possible to spin a polymer solution with a decreased amount of aqueous phase emulsified into the solution.

2. Co-Electrospinning

In a further aspect, fiber morphology can be varied by spinning from a multiphasic fiber-forming medium such as, for example, an emulsion, rather than from a solution or a dispersion. Advantageously, by using at least two-solvent systems having varying evaporation rates and miscibility, morphology of the resulting fiber can be controlled, wherein a preferential evaporation of the more volatile solvent causes the formation of outer surfaces or skins similar to those produced in, for example, a sausage casing process, where the less volatile liquid phase is entrapped and surrounded by a solidified polymer skin. Thus, the invention provides a method for making fibers of different morphologies, including, for example, flattened porous forms. The ability to control morphology of the fiber is useful in various medical applications, such as, for example tissue engineering, drug delivery, as well as non-medical application such as, for example, electronics. Another unexpected benefit of this invention is that due to the addition of aqueous phase, resulting fibers can be produced with small diameters, as compared to the fibers produced from a single-phase solution of identical polymer concentration.

In one aspect, co-spinning, for example co-electrospinning, can be performed by spinning more than one polymer dissolved in a polymer solution, for example a solution of polyurethane and poly(lactic acid). In a further aspect, co-spinning, for example co-electrospinning, can be performed by simultaneously spinning more than one polymer from more than one polymer solution, for example a solution of polyurethane and a solution of poly(lactic acid), using a dual needle system.

Accordingly, the co-electrospinning methods can provides a method of making a fiber from an emulsion comprising a first component including water, and a second component including a polymer dissolved in a solvent. In the method, a force is applied to the emulsion to extrude and separate the emulsion into a fiber. The force is preferably created by an electrostatic field, i.e., an electric force. In this method, the emulsion is preferably electrically conductive or includes electrically conductive materials. Other examples of the force include a magnetic force, an electromagnetic force, or the force of pressurized gas.

Apparatuses useful in this method for creation of the electrostatic field are known in the art such as, for example, electrospinners described by Fridrikh et al. and Bomat supra. These apparatuses employ the electric force for spinning the multiphasic fiber-forming medium of the invention. Another type of apparatuses employs a compressed gas as described by U.S. Pat. No. 6,520,425 by Reneker.

The multiphasic fiber-forming medium of the invention is an emulsion, such as, for example, a water/oil emulsion, a double emulsion or an emulsion in which particles are dispersed. In forming the emulsion, at least two components are mixed, wherein the first component (an aqueous phase or a hydrophilic component) has first evaporation rate, and the second component (an oil phase or a lipophilic component) has a second evaporation rate, such that the second evaporation rate is higher than the first evaporation rate.

By varying the ratio of components in the emulsion, desired morphology can be achieved as described below. In certain aspects, the first component and the second component are provided at a ratio, wherein the ratio is adapted to change morphology of the fiber and its diameter. Examples of fibers with various morphologies include flat fiber, round fiber, porous fiber and combinations thereof. It was observed for an exemplary PLA emulsion, the transition from round to porous fibers occurs in the range of from about 2 to about 5% volume fraction of aqueous phase in the emulsion. Above 5% volume fraction of aqueous phase, fibers with a flat-ribbon morphology are obtained.

In certain aspects, the first component comprises water and optionally, glycerol and poly(vinyl alcohol). In certain aspects, the first component comprises at most 40 vol % of the emulsion. In certain aspects, the first component comprises from about 5 to about 40 vol %, for example, from about 5 to about 20 vol % or from about 5 to about 10 vol %. In certain aspects, the first component comprises 2 to 5 vol %.

In certain aspects, the second component comprises at least 60% of the emulsion. In certain aspects, the second component comprises polymer dissolved in an organic solvent. Non-limiting examples of suitable polymers include poly(styrene), poly(urethane), poly(lactic acid), poly(glycolic acid), poly(ester), poly(alpha-hydroxy acid), poly(ε-caprolactone), poly(dioxanone), poly(orthoester), poly(ether-ester), poly(lactone), poly(carbonate), poly(phosphazane), poly(phosphanate), poly(ether), poly(anhydride), mixtures thereof and copolymers thereof. Further, one or more surfactants, emulsifiers, and/or stabilizers can be added to the emulsion for impacting properties of emulsion such as stability, consistency, etc. Depending on ratios of first component to the second component, the emulsion can be a microemulsion.

In certain aspects, the emulsion can comprise a third component such as for example, a bioactive agent, a cell, a particle, and/or a gel. The third component can be dissolved in either or both of the phases or it can be dispersed. Depending on the choice of the phase, the third component can be located inside or outside of the fiber. For example, if the third component is dissolved in the aqueous phase, upon forming of the fiber, it will be trapped insider, upon evaporation of the solvent of the second phase. Also, if the third component is dissolved in the second phase, upon forming of the fiber, it will be trapped in the outer skin of the fiber.

In certain aspects, the particle is a colloidal particle or a solid particle. Patterning the surfaces of fibers with particles has practical applications, for example, in tissue engineering where presentation of chemical and physical cues on degradable scaffolds allows a more precise control over cell-scaffold interactions.

In certain aspects, the colloidal particle has a diameter of from about 3 nm to about 10 micrometers and includes a polymer, an oxide, a nitride, a carbide, calcium silicate, calcium phosphate, calcium carbonate, a carbonaceous material, a metal, and a semiconductor.

An example of incorporation of solid particles is encapsulation silica nanoparticles (SNP) within polymeric fibers. The presence of SNP within the fibers was verified using SEM and BET measurements, which revealed the presence of a phase with a very high surface area (>50 m²/gM). Also, carbon nanotubes and magnetic particles are examples of solid particles suitable in this invention.

Non-limiting examples of surfactants include non-ionic surfactants such as, for example, PLURONIC, polyvinyl alcohol, poly(sorbate)(such as, for example, TWEEN-80 and SPAN-200, oleyl alcohol, glycerol ester, sorbitol, carboxy methoxy cellulose or an ionic surfactant such as, for example, sodum dodecyl sulfonate, sodum dodecyl benezene sulfonate, oleic acid, albumin, ova-albumin, lecithin, natural lipids, and synthetic lipids.

In certain aspects, the emulsion comprises water mixed with poly(vinyl alcohol) as the first components and poly(lactic acid) dissolved in organic solvent as the second component, and optionally, silicone oxide nanoparticle having a biomolecule attached to the nanoparticle's surface as the third component.

Using a multiphase, emulsified solution in electrospinning affords two controllable fiber characteristics, fiber diameter and surface morphology. This is accomplished by two principles arising from the emulsion system: increase in apparent viscosity and immiscible solvent templating effects.

First, an increase in the apparent viscosity of the solution allows for electrospinning of a lower concentration of the polymer in the compatible solvent. Lower viscosity solutions or solutions with low polymer concentrations tend to electrospray, forming polymer droplets rather than fibers at the grounded electrode. However, by adding additional phases as an emulsion, it is possible to increase the viscosity at the needle tip. This increase in viscosity allows for the formation of a more stable Taylor cone, and thus, produces fiber.

However, increasing polymer concentration does not allow for the formation of ultrafine fibers. It is widely reported that increasing polymer concentration in numerous polymer/solvent systems. However, by using a multiphase solution, the present methods are able to use polymer concentrations that typically electrospray in a one-phase solution. In addition, the methods of invention demonstrate an increase in shear thinning (decrease in viscosity) at the end of the Taylor cone, allowing for even finer fiber formation.

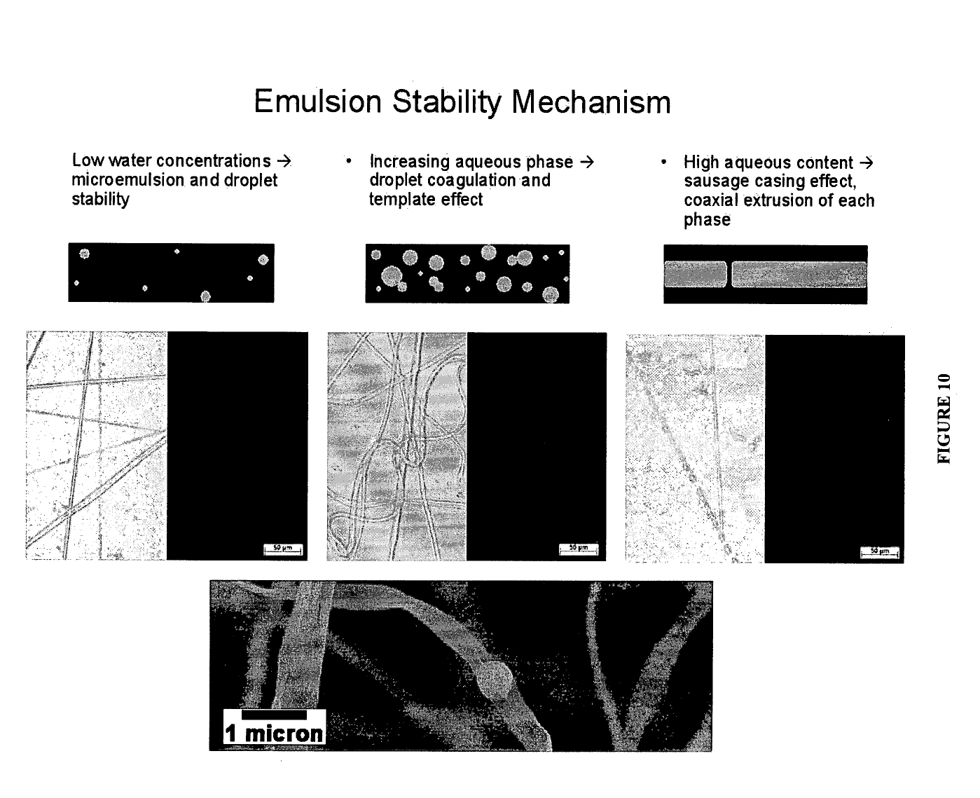

Second, emulsifying a second, immiscible phase into the ES solution allows for templating of the resulting fiber. Researchers have used similar techniques to produce hollow nanofibers by using a coaxial spinneret system. Similarly, the present methods are able to see the transition between solid, round fibers to porous fibers to flat/collapsed hollow fibers. Fiber surface morphology is dependant on the concentration of the immiscible phase. Round fibers are found when very little immiscible phase is emulsified in the polymer/solvent solution. Suspended droplets of the immiscible phase create pores as the concentration of the immiscible phase increases. Eventually, a concentration is reached where droplets coalesce during fiber formation, forming sausage-link-like structures that result in hollow nanotubes. Depending on the modulus/mechanical properties of the polymer of interested, fibers showed either hollow tube morphologies or collapsed, ribbon like structures. In these examples, high molecular weight, high modulus polymers (PLA and Polyox PEO) tend to provide collapsed ribbon structures, while elastomers (PU and PEVA) did not show ribbon-like morphology FIG. 10.

By increasing the apparent viscosity of the solution at the spinneret orifice, the present invention can electrospin polymer solutions that typically do not form fibers. As a result, the lower polymer concentrations produce smaller-diameter fibers.

Applications of this co-spinning technique include tethering growth factors to ECM proteins and patterning discrete parts of the scaffold with bioactive signaling molecules, combining different synthetic polymers to more closely match the mechanical properties of native tissue, localizing anti-thrombogenic agents in the graft, and delivering cells to discrete regions of the graft by including them in one of the co-spun fluid phases.

3. Spinning

In one aspect, the conduits of the invention can be prepared using conventional polymer fiber spinning techniques. In this aspect, the polymer need not be provided by electrospinning. By providing a polymer fiber and collecting the fiber on a rotating mandrel, conduits having a substantially tubular body comprising substantially circumferential polymer fibers; wherein the body has an exterior surface, an interior surface, and a lumen extending therethrough; and wherein the body has microscale features disposed at the interior surface can be provided.

In this aspect, however, the microscale features can be limited to larger diameters, widths, depths, and heights due to the relatively larger fiber thickness/diameter of the polymer fiber when provided by conventional polymer spinning techniques. In a further aspect, the present invention can exclude non-electrospinning techniques of providing a polymer fiber.

4. Collection of FIBERS

Using a rotating mandrel as a target, fibrous tubular conduits/scaffolds several centimeters in length, and from about 100 to about 150 μm in thickness can be spun in a matter of few minutes. Rapid fabrication of scaffolds can be vital for optimization of the system with respect scaffold characteristics (e.g., compliance, modulus) and cell interactions (e.g., attachment, proliferation). Additionally, the use of an aqueous phase in the fiber spinning process, allows for modification of the scaffold with additives, for example bioactive agents such as growth factors and peptides that can promote cell proliferation and differentiation or pharmaceutically active agents or pharmaceutically active agents.

One factor that can affect fiber alignment is the needle tip-target distance. By varying the composition of the emulsion, solidification of the polymer fibers can be accelerated, thus favoring shorter needle tip-target distances. By manipulating the speed of the rotating mandrel target in combination with these strategies fiber whipping can be significantly diminished to promote alignment of the electro-spun fiber.

In one aspect, the mandrel rotates at a constant rotational speed. A constant rotational speed can facilitate uniform conduit wall thickness. A constant rotational speed can also maintain a substantially constant polymer solution composition. In a further aspect, the mandrel can rotate at a non-constant speed. For example, the mandrel can be rotated at a speed that is increasing over time, at a speed that is decreasing over time, or a combination thereof.

5. Including Supplementary Materials

In one aspect, the methods of the invention can further comprise the step of co-electrospinning a supplementary material onto the rotating mandrel. In one aspect, the supplementary material can be, for example, collagen, fibrin, chitin, laminin, poly(ethylene glycol), or a mixture thereof. In a further aspect, the supplementary material can be at least one synthetic peptide, polysaccaride, proteoglycan, extracellular matrix component, or a mixture thereof. In various aspects, the supplementary material comprises polymer fibers. In further aspects, the supplementary material is nonpolymeric.

In one aspect, the electrospinning step and the co-electrospinning step are performed simultaneously. In a further aspect, the electrospinning step and the co-electrospinning step are performed sequentially. In one aspect, the spinning step and the co-spinning step are performed simultaneously. In a further aspect, the spinning step and the co-spinning step are performed sequentially.

The supplementary material can be provided in the present invention, for example, during the electrospinning process by adding the supplementary material to the polymer solution or emulsion or can be provided during the electrospinning process by separate solution in a dual needle system.

6. Including Additives

In one aspect, the methods of the invention can further comprise the step of co-electrospinning one or more additives onto the rotating mandrel. In one aspect, the one or more additives comprises a pharmaceutically active agent, for example, an antithrombogenic agent such as heparin. In this aspect, once the conduit so produced is implanted into a subject, the additive can then be released from the porous conduit into a subject. In this aspect, the conduit can serve as an additive, for example pharmaceutically active agent, delivery system. The one or more additives can be provided in the present invention, for example, during the electrospinning process by adding the additives to the polymer solution or emulsion.

In one aspect, the electrospinning step and the co-electrospinning step are performed simultaneously. In a further aspect, the electrospinning step and the co-electrospinning step are performed sequentially. In one aspect, the spinning step and the co-spinning step are performed simultaneously. In a further aspect, the spinning step and the co-spinning step are performed sequentially.

7. Creating Microscale Features

In one aspect, the negative relief of suitable micro-patterns can be transferred onto a thin (100 μm) Silastic master (Sylgard®). Suitable microscale features include the reservoirs, protrusions, wells, ridges, and grooves described herein and the like. The master can then be wrapped around the rotating mandrel and the fibers deposited onto this template. In a further aspect, the mandrel can be adapted to provide the negative relief of suitable micro-patterns in the absence of a Silastic master.

In conventional approaches, the use of synthetic polymeric vascular grafts can be limited by the thrombogenecity of most biomaterials. Efforts to reduce thrombogenecity, by seeding with endothelial cells, the natural non-thrombogenic lining of blood vessels, have been thwarted by flow-induced cell detachment. By creating well-defined micro-textured structures or patterns on a surface, fluid flow at the surface can be altered to create discrete regions of low shear stress. Due to reduced shear stress, these regions can serve as sanctuaries for endothelial cells and promote their retention.

8. Adhering Cells to the Interior

Numerous conventional methods to increase adhesion and retention of EC to a polymeric surface have been explored, including covalent modification (see Massia, S. P. and J. A. Hubbell, Human endothelial cell interactions with surface-coupled adhesion peptides on a nonadhesive glass substrate and two polymeric biomaterials, J Biomed Mater Res, 1991, 25(2): p. 223-42; Lin, H. B., et al., Endothelial cell adhesion on polyurethanes containing covalently attached RGD-peptides, Biomaterials, 1992, 13(13): p. 905-14; Walluscheck, K. P., et al., Improved endothelial cell attachment on ePTFE vascular grafts pretreated with synthetic RGD-containing peptides, Eur J Vasc Endovasc Surg, 1996, 12(3): p. 321-30; and Stachelek, S. J., et al., Cholesterol-derivatized polyurethane: characterization and endothelial cell adhesion, J Biomed Mater Res A, 2005, 72A(2): p. 200-12.), adsorption of native (see Walluscheck, K. P., et al., Improved endothelial cell attachment on ePTFE vascular grafts pretreated with synthetic RGD-containing peptides, Eur J Vasc Endovasc Surg, 1996, 12(3): p. 321-30; and Carr, H. M., et al., Endothelial cell seeding kinetics under chronic flow in prosthetic grafts, Ann Vasc Surg, 1996, 10(5): p. 469-75.) or engineered proteins (see Hsu, S. H., S. H. Sun, and D. C. Chen, Improved retention of endothelial cells seeded on polyurethane small-diameter vascular grafts modified by a recombinant RGD-containing protein, Artif Organs, 2003, 27(12): p. 1068-78.), prior seeding with smooth muscle cells (see Yu, H., et al., Smooth muscle cells improve endothelial cell retention on polytetrafluoroethylene grafts in vivo, J Vasc Surg, 2003, 38(3): p. 557-63.), use of pressure (see Chan, B. P., et al., In vivo performance of dual ligand augmented endothelialized expanded polytetrafluoroethylene vascular grafts, J Biomed Mater Res B Appl Biomater, 2005, 72(1): p. 52-63.) or electrostatic forces (see Bowlin, G. L., et al., In vitro evaluation of electrostatic endothelial cell transplantation onto 4 mm interior diameter expanded polytetrafluoroethylene grafts, J Vasc Surg, 1998, 27(3): p. 504-11.) to facilitate endothelial seeding, modification of endothelial cells (see Chan, B. P., et al., In vivo performance of dual ligand augmented endothelialized expanded polytetrafluoroethylene vascular grafts, J Biomed Mater Res B Appl Biomater, 2005, 72(1): p. 52-63.), and mechanical preconditioning of seeded cells (see Baguneid, M., et al., Shear-stress preconditioning and tissue-engineering-based paradigms for generating arterial substitutes, Biotechnol Appl Biochem, 2004, 39(Pt 2): p. 151-7; and Dardik, A., A. Liu, and B. J. Ballermann, Chronic in vitro shear stress stimulates endothelial cell retention on prosthetic vascular grafts and reduces subsequent in vivo neointimal thickness, J Vasc Surg, 1999, 29(1): p. 157-67.).

In one aspect, one or more cells can be adhered to the interior surface of the conduits of the invention. Cells such as bovine aortic endothelial cells (BAEC) readily adhere to the fibrous structure of the conduits without the need for any pre-conditioning. In this non-chemical approach, polymer surfaces can be patterned using micro-fabrication techniques, to possess discrete well-defined noncontiguous areas (zones). By presenting the surface with well-defined microscale features (topography), fluid flow at the surface can be altered to create discrete regions of lowered shear stress. These regions of low-shear stress can serve as sanctuaries for the retention and survival of endothelial cells.

In one aspect, the methods of the invention can further comprise adhering at least one cell to the interior surface. The at least one cell, in one aspect, can comprise at least one endothelial cell, nerve cell, Schwann cell, glial cell, or stem cell, or a mixture thereof.

Cells can be provided for adhesion at the interior surface using a bioreactor such as provide in FIG. 6. In a typical procedure, a PU scaffold 4 cm long can be endothelialized by seeding the lumen with porcine aortic endothelial cells at sub-confluent density (105 cells/cm²) and cultured until the lumen is covered with a confluent monolayer of EC. In order to facilitate the growth of EC, the culturing can be done in presence of FGF-2, a growth factor know to enhance the proliferation of endothelial cells and fibroblasts. A 2 cm portion of endothelialized graft can then be placed in a dual perfusion bioreactor (FIG. 6) and subjected to a pulsatile flow (165 pulses/minute, WSS 7 dynes/cm² and 14 dynes/cm²) for a period of 24 hours, while the remaining 2 cm can serve as a positive control (no flow). The flow rate needed to provide the requisite WSS can be calculated as per the Hagen-Poiseuille equation (Equation 1). At the conclusion of the procedure, the center of the scaffold can be dissected and a 1 cm long portion harvested, half of which can be used for visual observation after fixing and staining with DAPI, while the other half can be assayed for cellular activity using the MTT mitotic activity assay. The values obtained are normalized to the control to get an estimate of percent EC retention after flow.
τₘₑₐₙ=(4 μQ)/πR³ Equation (1)

For a near-circular or elliptical lumen, assuming parabolic laminar flow conditions, the temporal and spatial average shear stress (τₘₑₐₙ) can be determined using the above equation, where Q is the flow rate in ml/sec, μ is the viscosity of blood, and R is the inner radius of the lumen.

A retention value above 80% is typically considered suitable for in vivo evaluation in a subject, for example, Yucatan pigs.

9. Adhering Cells to the Exterior

In one aspect, the methods of the invention can further comprise adhering at least one cell to the exterior surface. In one aspect, the at least one cell can comprise at least one smooth muscle cell, at least one transfected fibroblast, or a mixture thereof.

Vascular SMC and aortic endothelial cells can be isolated from a subject, for example, from a Yucatan miniature pig, using well established protocols. After seeding the lumen of the scaffold with EC, the exterior of the scaffold can be seeded with vascular SMC using a spinner flask seeding system and cultured using media and conditions described by Niklason et al., Functional Arteries Grown in Vitro, Science, 1999, 284, 489-493. After 1-week in culture, the scaffold can be transferred to bioreactors (FIG. 6) and cultured under pulsatile flow conditions for an additional period of from about 2 to about 3 weeks to ensure maturation of the SMC layer. The grafts can then be characterized histologically and using immunohistochemistry for α-actin, tenascin-C, elastin and type-I collagen. Further, the contractile behavior of the grafts in response to known pharmacological agents such serotonin and endothelin-1 can be assessed in a qualitative manner (contraction/no contraction).

When grafts are engineered for implantation, autologous cells can be used to avoid rejection of the graft.

Typically, a number of different methods can be used to attach smooth muscle cells, fibroblasts, or other cell type to the conduit surfaces. First, traditional tissue engineering methods of static seeding or dynamic seeding in spinner flasks may be used.

For example, a conduit can be seeded statically by suspending cells at a high concentration (>500,000 cells/ml) in culture media and applying the suspension directly on the scaffold. This technique allows cells to settle onto the scaffold and attach to the polymer matrix. However, this technique typically yields non-uniform cell distribution throughout the scaffold.

In order to increase seeding efficiency, a number of dynamic seeding protocols have been developed by researchers (see K. J. L. Burg et al., Comparative study of seeding methods for three-dimensional polymeric scaffold, J Biomed Mater Res., 2000, Sep. 15;51(4):642-649). For example, dynamic seeding methods can use slowly rotating spinner flasks (˜1-10 RPM) to seed polymeric scaffolds with even distributions.

In order to improve the efficiency of cell seeding, the surfaces of a synthetic conduit can be modified to improve cellular retention and attachment. In one aspect, one or more highly porous layers of PLGA (either produced by electrospinning, solvent evaporation techniques, or other scaffold-forming process) can be wrapped or formed around the outside of the tubular conduit. Such a layer can be bonded either by using chemical-crosslinking agents (chemically), solvent or thermal techniques (physical), or a combination of the two. Furthermore, the additional layer(s) may be surface modified (treated with NaOH to increase hydrophilicity in the case of PLGA meshes) to allow for greater cellular attachment. An additional porous layer can allow for greater surface area for cellular attachment (e.g., through either static or dynamic seeding methods) and proliferation.

Another method of cell seeding that can be used in connection with the present invention is the contraction of a cell-bearing collagen gel around the tubular conduit. Such a method can yield very high cell seeding efficiency. See Experimental Examples.

In a further aspect, fully confluent cell sheets may also be used to seed the interior or exterior of the scaffold. Cells may be grown in subculture and released as full sheets using either enzymatic, ultrasound, chemical, or thermal techniques (as described in, for example, N. L'heureux et al., A completely biological tissue-engineered human blood vessel, FASEB, 1998, 12, 46-56; Y Hayashida, et al., Ocular surface reconstruction using autologous rabbit oral mucosal epithelial sheets fabricated ex vivo on a temperature-responsive culture surface, Investigative Ophthalmology & Visual Science, 2005, 46(5), 1632-39; and T Shimizu, et al., Cell sheet engineering for myocardial tissue regeneration, Biomaterials, 2003, 24, 2309-16.). Briefly, cell sheets can be produced by growing cells in supplemented media to increase their ECM production. The increase in ECM production aids in removal (using tools, ultrasound, enzymes, or a combination) by providing a more substantial material to work with. Temperature dependent hydrophobicity/hydrophilicity properties of certain polymers (e.g., poly(N-isopropylacrylamide) (PIPAAm)) may also be used to release cell sheets by lowering the culture surface below its critical temperature. Using these techniques, endothelial cell sheets may be applied directly to the lumen and held in place to allow for cellular attachment. Similarly, SMC or Fibroblast sheets may be wrapped around the exterior of the scaffold as a means of seeding the construct.

### E. METHODS OF USING SYNTHETIC CONDUITS

In one aspect, the invention is directed to a biocompatible tubular conduit, prosthesis, or scaffold, which, when implanted into a subject, can serve as a functioning repair, augmentation, or replacement body part or tissue structure.

In a further aspect, the present invention provides a method of implanting a vascular prosthesis comprising the steps of providing the prosthesis produced by the methods of the invention, and implanting the prosthesis into a subject.

In a further aspect, the present invention provides a method of implanting a stent comprising the steps of providing the stent produced by the methods of the invention, and implanting the stent into a subject.

In a further aspect, the present invention provides a method of implanting a nerve regeneration scaffold comprising the steps of providing the scaffold produced by the methods of the invention, and implanting the scaffold into a subject.

### F. EXPERIMENTAL EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

1. Electrospinning

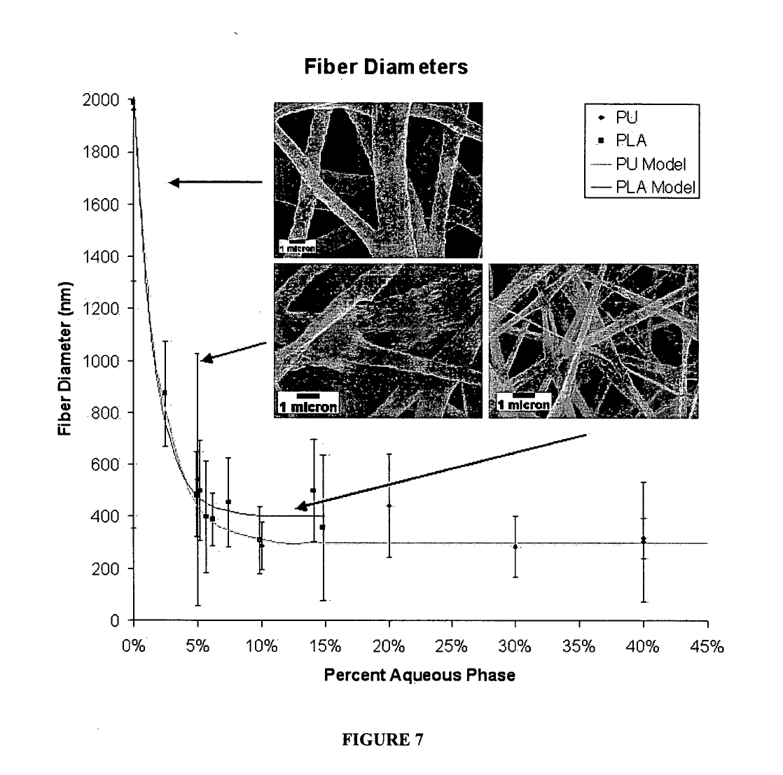

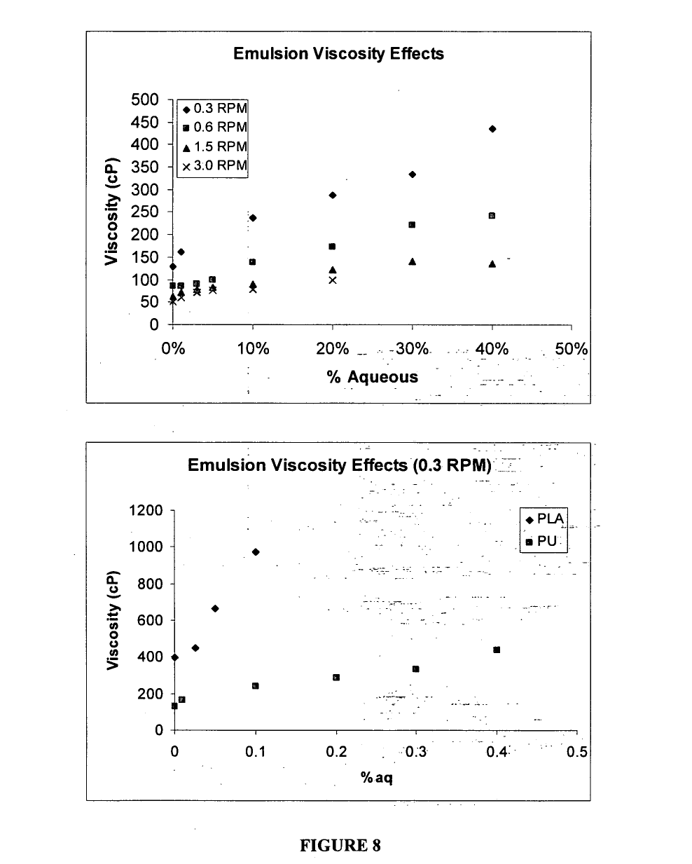

A series of solutions using 15% (w/w) PU solution with varying amounts of aqueous phase was electrospun (17 kV applied voltage, 20 cm tip-to-target distance, 0.1 ml/min, 16 gauge needle) to produce the fiber diameter versus percent aqueous phase curve shown in FIG. 7. The emulsion mechanism has the most drastic effects in the low aqueous concentration ranges (0-5%), consistent with rheological data that suggests that the most dramatic effect in shear thinning comes with small additions of the second immiscible phase. There is an order of magnitude decrease in average fiber diameter (from 1960 nm to 540 nm) with the addition of 5% (w/w) of poly(vinyl alcohol) (PVA) solution. Without wishing to be bound by theory, it is believed that one possible mechanism for this large decrease in fiber diameter is the fact that the emulsion exhibited enhanced shear thinning at the tip of the “Taylor cone,” allowing for a greater reduction in the cone diameter. Towards the end of the “Taylor cone” the less volatile aqueous phase occupies a greater volume fraction of the jet, allowing for greater thinning/deformation of the cone compared to the highly viscous polymer/organic solvent gel.

A similar experiment was performed using a poly(1-lactic acid) (PLA) system. Using a base solution of 2% (w/w) PLA dissolved in chloroform, varying amounts of PVA/water and N-methylpyrrolidone (NMP) were emulsified with the polymer solution and electrospun (25 kV applied voltage, 15 cm tip-to-target distance, gravity fed spinneret, 16 gauge needle). Diameter versus percent aqueous phase is shown in FIG. 7. The PLA system showed a similar order of magnitude decrease (from 2000 nm to 490 nm) with the addition of just 5% aqueous phase.

For both systems, the decrease of fiber diameter with increasing aqueous content leveled out to 300 nm and 400 nm for PU and PLA, respectively. There was an upper limit to the amount of aqueous phase that could be added to the solutions since high aqueous containing solutions tended to electrospray. However, this asymptotic behavior of the fiber diameter indicates that the mechanism is largely a shear thinning mechanism, rather than a concentration effect. While apparent viscosities of the solutions increased with aqueous concentration, all emulsions showed a limiting shear thinning viscosity value.

PLA and polyurethane (PU) emulsions were tested using a Brookfield Viscometer (Model LVDV-II+, Middleboro, Mass.) with a cone and plate spindle (model CPE-40, 0.8° cone spindle, 0.5 ml sample volume) at room temperature. Rheological data obtained confirmed two principles of our proposed mechanism. An increase in apparent viscosity allowed for the electrospinning of low polymer concentrations while more pronounced shear thinning at high shear rates allowed for the formation of thinner fibers.

Samples of 6% PU (w/w) dissolved in THF/chloroform (equal volumetric ratios of each solvent) were emulsified with varying amounts of 10% PVA/water (w/v). FIG. 8 shows how increasing aqueous content of the solution increased the viscosity of the electrospinning solution. A 10% aqueous emulsion had over a two-fold increase in apparent viscosity of the solution (128.7 cP to 435.2 cP) at the slowest shear rate (0.3 RPM). This dramatic increase in apparent viscosity explains how the present methods electrospin, rather than electrospray, dilute polymer solutions. Similar results were obtained with the PLA system.

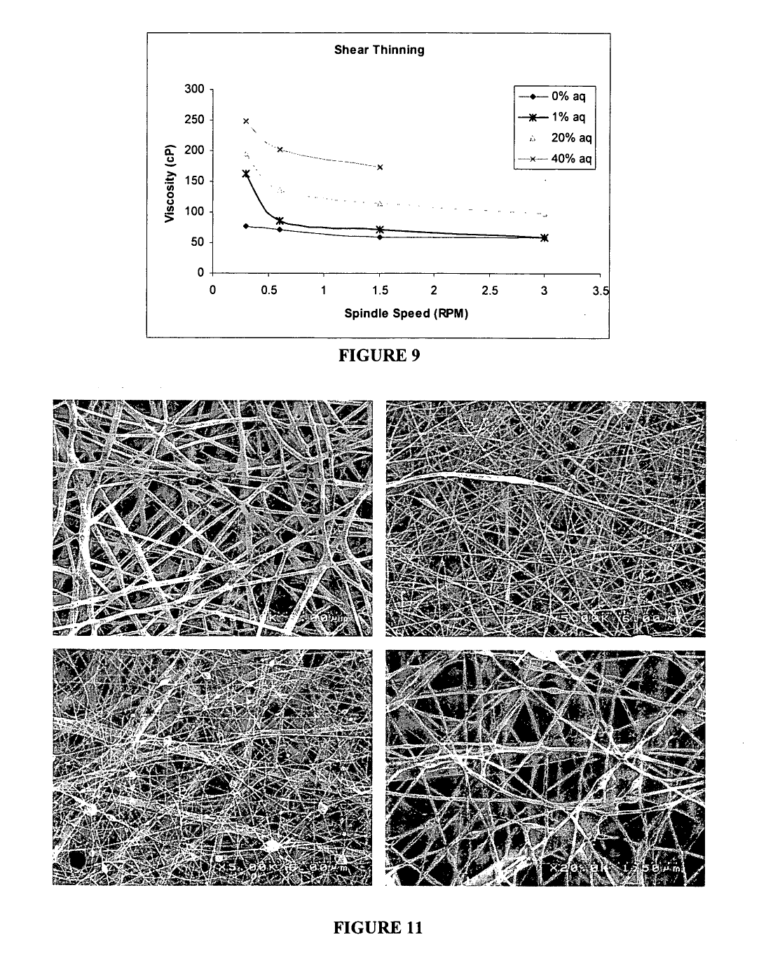

Shear thinning was tested using four different spindle rotational speeds (0.3, 0.6, 1.5, 3.0 RPM) at a number of different aqueous contents. For all aqueous concentrations, shear thinning was most pronounced at slower shear rates (i.e., the transition between 0.3 RPM and 0.6 RPM). As spindle rotational speed increased, the viscosity of the solution approached a limiting value (FIG. 9).

In addition to changing the rheological properties of the electrospinning solution, a secondary effect of adding multiple phases to the solution was that the less volatile liquid phase acted as a template during fiber solidification and formation. This effect produced fibers with varying morphologies ranging from round to porous to ribbon-like.

This effect was most evident in the PLA system. PLA fibers spun with low aqueous concentrations (<5% by volume) were predominately round in morphology. However, as the amount of aqueous phase increased in this range, fiber porosity increased. Without wishing to be bound by theory, it is believed that these pores were likely formed as the PLA solidified around the aqueous droplets during the electrospinning process. As the aqueous phase evaporated after the fibers were formed, they left behind open pores in the polymer matrix.

As aqueous concentration was increased above 5%, the appearance of ribbon-like fibers become more predominant. The likely mechanism for the formation of these fibers is due to the collapse of hollow PLA tubes. As the aqueous phase is increased, there is a greater volume fraction of water during the fiber formation process, which leads to a greater templating effect of the aqueous phase. This theory was tested by dispersing colloidal silica in the aqueous phase. Due to silica's hydrophilicity, the silica particles could be used to track the migration of the aqueous phase. The silica particles were sequestered in the polymer matrix and took on a pearl-chain configuration as particles were lined up in close proximity to one another.

While ribbon formation did not dominate at high aqueous concentrations for the PU and PEVA electrospun fibers, it is believed that the elastic properties of the polymer contribute to the amount the aqueous phase can act as a template during fiber formation. Both PU and PEVA have strong elastomeric properties. This recoverable elastic deformation of the polymer could have prevented the collapse of the hollow tubes. PLA has a much higher elastic modulus and less recoverable deformation. In fact, many of the PLA ribbons appear like they were split polymer tubes.

In addition, solvent compatibility also plays a role in this process. The particular PU used in this example was only dissolvable in THF, which is fully miscible with water. As a result, an additional organic solvent, chloroform in this case, was used to create an emulsion. However, the partitioning and phase separation of the aqueous phase was not fully studied and may have contributed to the round fibers seen with most of the PU samples.

For polymers that did not exhibit flat fiber morphology at high aqueous concentrations (PU, PEVA, and PVA not shown), larger droplets of water were encapsulated. The presence of the water phase was confirmed by labeling the water phase with fluoroscein. These features were visible on an optical microscope, indicating that the water droplet size was much larger than the actual polymer fiber diameter in that particular PEVA system.

2. Co-Spinning

Co-spinning scaffolds with both a degradable/biologically remodelable and non-degradable polymer using a two-needle cospinning approach was investigated. Scaffolds containing both PU and bovine type I collagen (electrospun out of a 40 mg/ml solution in 1,1,1,3,3,3-hexafluoro-2-propanol) have been produced using the methods of the invention (FIG. 11). By cospinning PU and collagen into the scaffold, it is possible to utilize strengths of both materials. PU provides a strong, elastic framework for the graft, lending it immediate mechanical integrity as well as compliance. Collagen provides a natural extracellular matrix (ECM) that can aid in cellular attachment, proliferation, and differentiation, but typically lacks adequate mechanical properties to be useful or functional. The addition of additional ECM proteins or other synthetic polymers allow further tuning of the physical and chemical properties of the conduit.

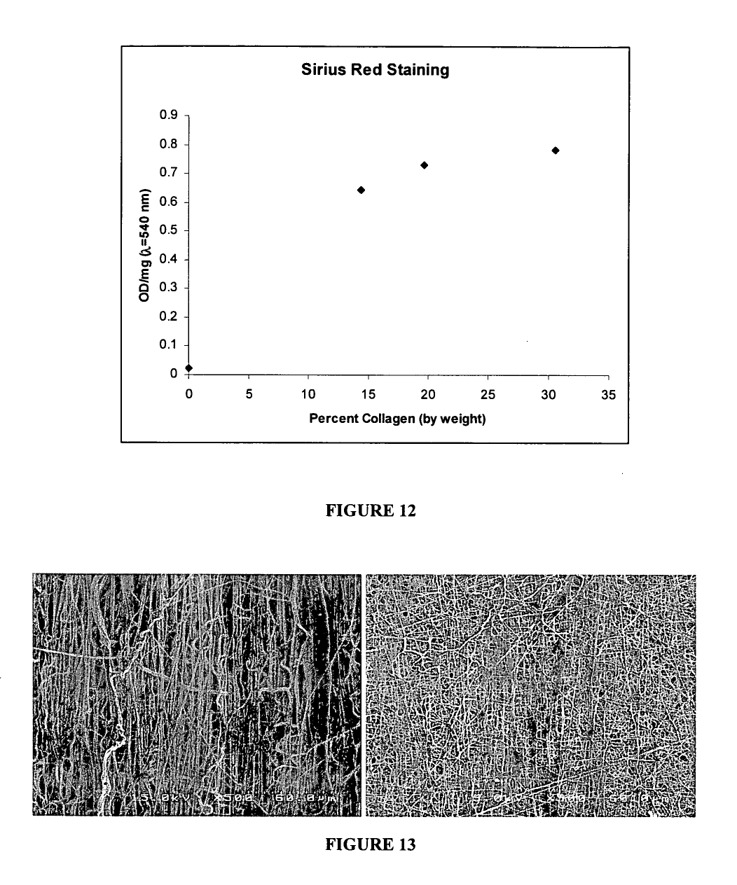

The presence of cospun collagen was confirmed both by SEM (as seen in FIG. 11) and spectrophotometrically. Samples containing different weight percentages of collagen were prepared by varying the flow rate of each polymer solution. Samples ranging from 0% collagen to 30% collagen were prepared. Scaffolds were fixed with gluteraldehyde to stabilize collagen fibers. Samples were subsequently stained with Sirius red/picric acid solution and rinsed to remove excess dye. Dye bound to the collagen fibers was solubilized in NaOH solution overnight and the resulting supernatant was analyzed spectrophotometrically. Optical density/absorbance was measured at 540 nm and normalized to sample mass. As expected, normalized optical density of the solubilized dye increased with collagen content (FIG. 12).

While not wishing to be bound by theory, it is believed that the increased interaction between multiple phases can create a higher viscosity than the component parts individually. This phenomenon can be particularly evident in the polyurethane/chloroform:THF (1:1) system of the present methods (see Table 1). Conventional methods require a polymer concentration of at least 12% (w/w) to produce fibers—lower polymer concentrations (5%, 10%, 1%) electrospray under the same conditions. In contrast, with the emulsion technique of the invention, it was possible to spin a 7.5% (w/w) PU solution with as little as 5% aqueous phase emulsified into the solution.

TABLE 1

| | | | | | | Polymer |
|---|---|---|---|---|---|---|
| | | | | Tip-to- | | concentration |
| | Organic | Phase | Applied | target | Molecular | (in organic |
| Polymer | Solvent | compatibilizer | Voltage | distance | Weight | solvent) |
| poly(l-lactic | CHCl₃ | NMP | 25 kV | 15 cm | 300 kDa | 2% |
| acid) | | | | | | |
| polyurethane | CHCl₃/THF | none | 17 kV | 20 cm | 130 kDa | 6% |
| poly(ethylene | CH₂Cl₂ | NMP | 25 kV | 15 cm | 70 kDa | 7.5% |
| co vinyl | | | | | | |
| acetate) | | | | | | |

3. Collection of Fibers

Researchers have been able to align electrospun fibers over short distances (<5 mm) using rotating collecting targets with narrow collecting surfaces and high rotational rates (X. M. Mo et al.; A. Theron, et al.) or by using a multiple electrode configuration (D. Li et al.). The present invention produces tubular constructs with fiber alignment over 5 cm using a different approach. Polymeric fibers are collected on a mandrel rotating at 7500 rpm and being translated laterally by an additional electric motor. The mandrel was grounded opposite a charged needle/polymer solution at 17 kV. Alignment is achieved by using short (˜5 cm) electrospinning tip-to-target distances (FIG. 4). Without wishing to be bound by theory, it is believed that this shorter distance reduces the time of flight of the polymer fiber, thereby reducing the whipping motion of the Taylor cone. Substantially aligned polyurethane fibers (FIG. 13) were collected using the electrospinning apparatus (FIG. 5).

Alignment of polymer nanofiber was confirmed using a Hitachi S-4200 Scanning Electron Microscope (SEM). Samples prepared were composed of a polyurethane (PU)/poly(vinyl alcohol) (PVA) solution using an emulsion system (JC Sy, I-W Chen, VP Shastri, 2004 & Patent application No. 60/556,637). The emulsion system allowed tailoring of fiber diameter as well as electrospinning viscosity.

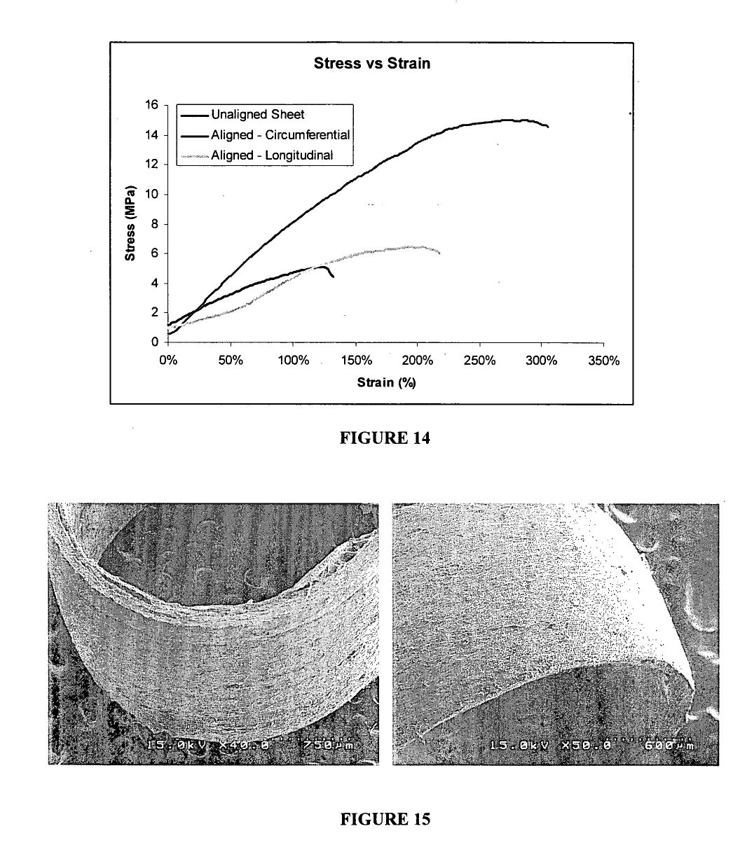

Mechanical data was also collected to confirm the alignment of the fibers (representative curves displayed in FIG. 14). A Shimadzu AGS-J universal tester was used to generate stress-strain curves of an unaligned sheet of PU and the aligned scaffold. For the aligned scaffold, samples were prepared to test the mechanical properties in the longitudinal and circumferential directions of the scaffold. Mechanical testing revealed that the elastic modulus of the randomly oriented PU fibers was similar to the longitudinal direction of the aligned scaffold (3.60±0.09 Mpa for the randomly oriented fiber and 3.37±0.23 MPa for the longitudinal direction). Aligned samples that were uniaxially loaded in the direction of the PU fibers had an elastic modulus of 7.89±0.10 MPa, a more than two-fold increase in elastic modulus.

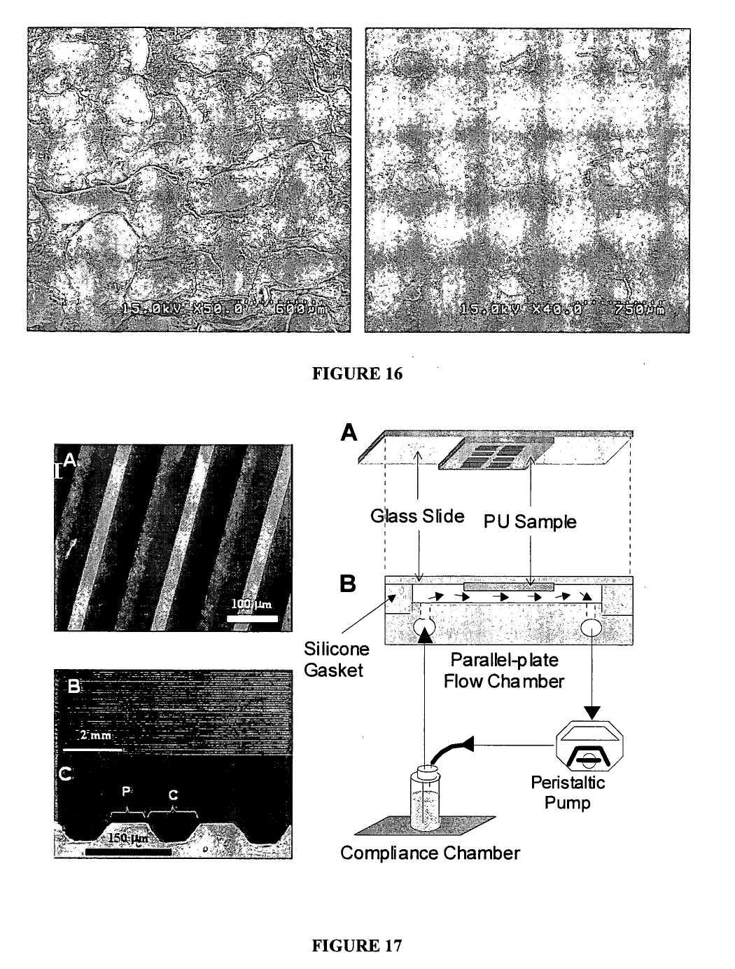

Four-centimeter tubular scaffolds were produced (FIG. 15) using the electrospinning apparatus. First, textured sheets of the polymeric fiber were produced using standard electrospinning techniques onto either wire or plastic meshes (FIG. 16). Electrospinning conditions were adjusted so that the collected fiber retained the desired topology of the template target material. Fiber mats are removed from the target template and carefully cut to form the lumen of the scaffold. Next, 3 mm diameter stainless steel mandrels are wrapped with a layer of aluminum foil to assist in the removal of the scaffold. The textured sheet, cut to fit as a single layer, is wrapped around the foil/mandrel. This target is then loaded into the electrospinning apparatus and aligned PU fibers are electrospun over the mandrel.

4. Creation of Microscale Features

By creating an array of well-defined micron size regions, an array of cell reservoirs can be created that can serve as anchorage points for the retention of a whole endothelial monolayer. In this example, an array of tens of micron-sized channels (i.e., grooves) was created on a PU film surface (FIG. 17, left panel) by solution casting of PU on a silicon wafer containing the negative features, i.e., an array of ridges. The channels were arranged in 4 rectangular grids (4 mm×5 mm) composed of 31 channels per rectangle. The dimension of each channel was 32 μm deep, 42 μm wide at its base and 95 μms wide at it top (i.e., plateau-to-plateau distance), with channels walls inclined at an angle of 54.7 degrees to the channel bottom.

5. Flow Studies

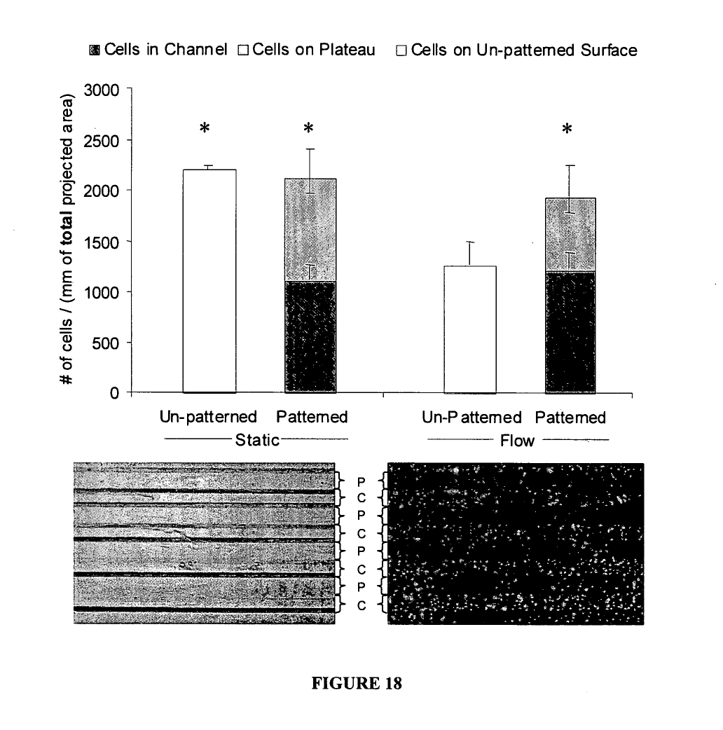

Retention of bovine aortic endothelial cells (BAEC) on micro-channel patterned polyurethane (PU) films in parallel flow chambers (FIG. 17, right panel) under laminar flow conditions was examined. When exposed to a very high shear stress of 60 dynes/cm² for one hour, which is approximately ten times the nominal arterial wall shear stress (WSSₐᵣₜₑᵣᵢₐₗ˜6.8 dynes/cm²), BAEC were extensively and preferentially retained in the channels over the normal surface (FIG. 18). In unpatterned PU controls exposed to flow, some regions were completely denuded of cells and the total cell density was decreased by 42% (FIG. 18, a<0.005). In contrast, when patterned PU was subjected to the same flow, the total cell density only decreased by 8% (FIG. 18, a>0.05).

6. Effect of Micro-Pattern Dimensions on Fluid Flow

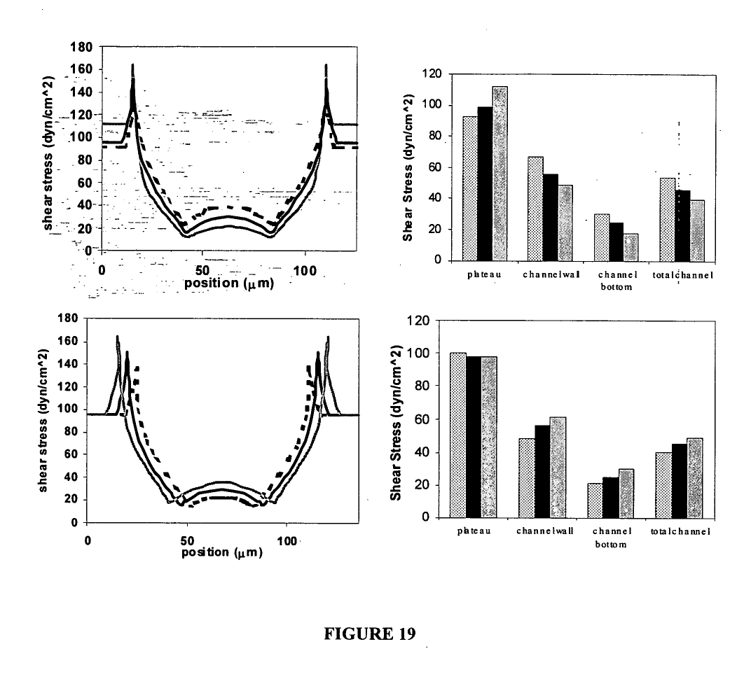

Computational fluid dynamics (CFD) can rapidly explore the effects of changing channel geometry and shape and thus aids in the rational design of patterned surfaces. Using the same fluid boundary conditions and fluid velocity, models were created that investigated the effects of increasing or decreasing overall channel depth (FIG. 19, top panel) and width (FIG. 19, bottom panel). The depth of the channel was varied from that of the physical model (32 μm, FIG. 19, top left, black line), by either increasing (40 μm, FIG. 19, top left, gray line), or decreasing (24 μm, FIG. 19, top left, dashed line) by 8 μm or 25% of the original depth, without altering the width of either the overall channel, or the bottom surface. Increasing the channel depth, and the inclination of the channel wall, increased both the peak shear stress at the plateau-channel junction and the average plateau shear stress, while decreasing the shear stress throughout the channel (FIG. 19, top right). Furthermore, when the width of the bottom surface of the channel was varied from the actual width of the physical model (42 μm, FIG. 19, bottom left, black line), by either increasing (52 μm, FIG. 19, bottom left, gray line) or decreasing (32 μm, FIG. 19, bottom left, dashed line) by 10 μm, without altering any other aspect of the geometry or the fluid flow conditions, increasing the channel width increased the peak shear stress at the plateau-channel junction but not the average plateau shear stress, while increasing the shear stress throughout the channel (FIG. 19, bottom right). These studies indicate that the geometry of the micro-channels (or micro features) effectively alters fluid-induced shear stress on biomaterial surfaces in a predictable manner, independent of material bulk properties as no temporal changes in PU films surface was observed during and after the flow studies.

7. Creating Microscale Features

Well-defined micro-textured polyurethane (PU) surfaces consisting of arrays of parallel 95-micron wide and 32-micron deep channels were created using an etched silicon template and solvent casting techniques. Based on computational fluid dynamics, relative to unpatterned surfaces exposed to a flow of 60 dyn/cm², the average local shear stress in the channels was 46 dyn/cm², which represented a 28% reduction in shear stress. When PU surfaces pre-seeded with endothelial cells (EC) were exposed to the same bulk flow rate, EC retention was significantly improved on the micropatterned surfaces relative to unpatterned surfaces (92% vs. 58% retention).

8. Optimization of PU Film Surface Micro-Topography and Flow Studies (Prophetic)

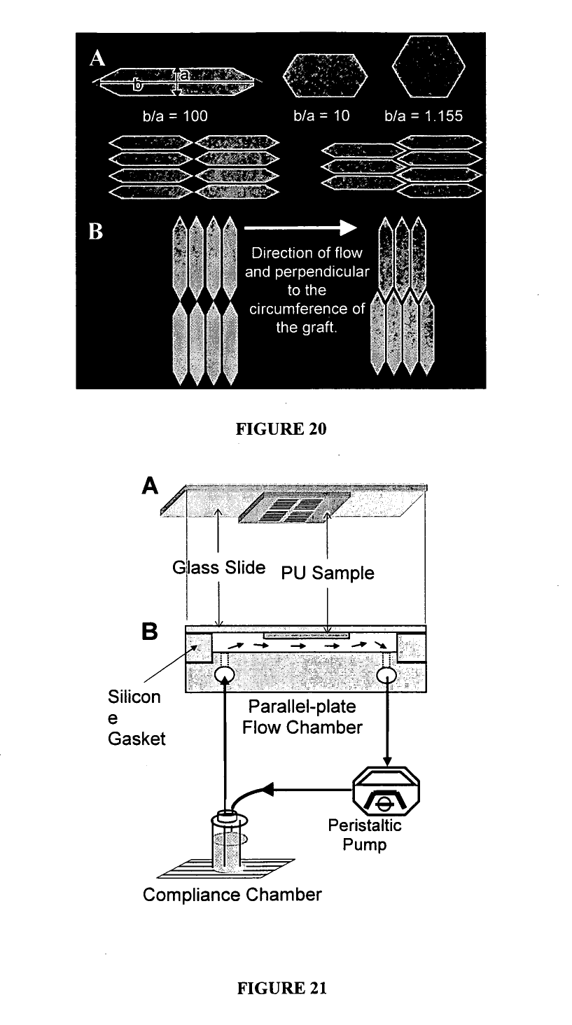

The array of channels used to demonstrate the feasibility of shielding EC in fluid-induced shear stress environment in essence is an elongated hexagon (FIG. 20 A). Two parameters can be varied (1) the ratio of the major/minor (b/a) axes of the hexagon and the spacing of the hexagonal structures. Three b/a ratios can be studied, 100, 10, 1.155 (regular hexagon). To minimize the potential geometric variations, the following four channel arrangements shown in FIG. 20 B can be explored. Since the channel depth of 32 μm yielded the expected outcome in the preliminary studies, a similar depth can be explored.

For the flow studies, silicon wafers with the negative image of the designed geometry can be fabricated using micro-fabrication and then used as templates for solution casting PU films. Porcine aortic endothelial cells can be harvested as per published protocols and seeded on PU films and cultured under standard conditions of 5% CO₂ in DMEM supplemented with 10% fetal bovine serum. Twenty-four hours post seeding, the films can be assembled onto glass substrates in a parallel flow chamber assembled and placed in an incubator, and subjected to flow rates to yield shear stress of ˜7 dynes/cm² (physiological) and 14 dynes/cm², for a period of 2, 8 and 24 hours (see Equation 2). The retention of cells can then be quantified by analysis of fluorescent images of cells after fixation and staining with DAPI, a nuclear stain. A percent retention of over 80% can be acceptable for translation of the micro-topography to the luminal wall of the tubular PU scaffolds.
τ=6 Qμ/wh² Equation (2)

Where τ is shear stress in dynes/cm², Q is flow rate in ml/s, μ is the viscosity of culture media (1 cP), w is width of the parallel plates (2 cm), and h is the spacing between the parallel plates in cm (0.0490 cm).

9. Animal Studies (Prophetic)

Four Yucatan pigs can be used for these studies, as they are well-established models and have physiological characteristics that closely mimic humans. Briefly, the pig will be anesthetized and prepped using sterile techniques and the engineered graft can be placed in the right sephanous artery. The fate of the graft can be followed for the duration of 1 month or failure of the graft, whichever comes earlier. The fate of the graft will be assessed on a weekly basis using Doppler ultrasound and imaged using digital subtraction angiography before explantation. The animal will be euthanized, and the graft will be explanted for histological evaluation (e.g., inflammation, thrombosis).

10. Contraction of a CELL-Bearing Collagen Gel

A synthetic conduit containing 30% type I collagen on the exterior of the conduit was sealed by placing Teflon plugs at each end of the tube. The scaffold was crosslinked in gluteraldehyde vapor for 4 hours at 37° C. The conduit was then removed from the gluteraldehyde vapor, and any unreacted gluteraldehyde was blocked by soaking the conduit in 0.1M glycine overnight. The conduit was subsequently washed using saline solution, sterilized in 70% ethanol, and incubated overnight in cell media containing serum. The construct was then placed in a mold, roughly 1 cm in diameter. A solution containing 1 million SMC/ml, 1 mg/ml type I rat tail collagen, 1× Hanks Buffered Saline Solution, 4 g/L glucose, 0.35 g/L sodium bicarbonate, and a predetermined amount of 1N NaOH to neutralize the acetic acid from the rat tail collagen solution to bring the pH of the mixture to 7.4, was poured into the mold. The conduit was then placed in a 37° C. incubator with 5% CO₂ to allow the collagen to gel. After 24 h, the collagen gels had contracted around the conduit, at which point, the conduit was transferred into a bioreactor or static culture.

11. Endothelial Cell Retention

To address the problem of endothelial cell loss from pre-seeded synthetic vascular grafts, non-chemical modification of the graft surface was explored. Without wishing to be bound by theory, it is believed that by creating well-defined micro-patterns on a surface, fluid flow at the surface can be altered to create discrete regions of lowered shear stress. Also without wishing to be bound by theory, it is believed that, due to reduced shear stress, these regions will serve as sanctuaries for EC and promote their retention. By creating a simple surface micro-architecture composed of an array of channels, local regions of lowered shear stress are produced, leading to improved EC retention.

a. Materials and Methods

Preparation of patterned template. A negative impression of the desired pattern of parallel channels was created on a 3″ silicon wafer using standard lithography techniques. The desired pattern was generated using AutoCAD-2000 and then transferred to a Ferrox mask. The wafer was then coated with AZ 5200-E positive photoresist (AZ Electronic Materials, Somerville, N.J.) and the desired pattern was then transferred to the resist surface by exposing the resist layer to UV radiation via a Ferrox mask bearing the desired pattern to depolymerize the resist in the exposed regions. The depolymerized resist was then removed by solubilization in a AZ 917 MIF developer (AZ Electronic Materials) and the exposed regions of the wafer were coated with a nitride layer by vacuum deposition. The unexposed resist was then removed using an AZ Kwik Strip stripper solution (AZ Electronic Materials) and exposed silicon surface was subjected to anisotropic etching using potassium hydroxide.

Preparation of micro-patterned polyurethane (PU) films. The silicon wafer template was used to pattern medical grade PU films by a solvent casting technique. In brief, a warm (45° C.) solution of segmented polyether urethane (ST1882, Stevens Urethane, Easthampton, Mass.) in tetrahydrofuran (Aldrich, St. Louis, Mo.) (75 mg/ml, company, city, state) was deposited on silicon wafer templates in a drop-wise manner until complete surface coverage was achieved. The film was air-dried for 12 hours and released from the silicon substrate by soaking in isopropanol, which also served to remove residual tetrahydrofuran. Non-patterned PU films were made by a similar casting procedure on un-patterned 3″-silicon wafer substrates. PU films were sterilized by immersion in 70% ethanol for 30 minutes and then dried overnight under the UV light of a laminar-flow cell culture hood.

Assembly of PU films and EC seeding. Using aseptic techniques, PU films were mounted on the center of autoclaved glass slides (#12-550B, Fisher Scientific, Fairlawn, N.J.) using autoclaved high-vacuum grease (Dow Corning, Midland, Mich.) such that the length of the channels (major axis) would be parallel to the direction of flow as shown in FIG. 21. The film-slide assemblies were placed in 10 cm Petri dishes (Fisher) and the PU surface was coated with a 100 μL drop of fibronectin (100 μg/ml, BD) solution in PBS (Gibco, Life Technologies Inc., Carlsbad, Calif.) and air-dried for 1 hour. Once the drop had evaporated, the PU-slide assembly was bathed in culture medium consisting of DMEM (Cellgro, Herndon, Va.), 10% FBS (Hyclone, Logan, Utah) and 1% penicillin/streptomycin (Gibco) and left in the incubator for 12 hours until cell seeding. Bovine aortic endothelial cells (BAEC) at passage 7 to 9 were seeded at 4×10⁵ cells per 16 mm×16 mm square 24 hours prior to the flow experiment. BAEC on all substrates exhibited cobblestone morphology typical of endothelial cells in a confluent monolayer.

Flow studies. The flow studies were carried out using a closed-loop perfusion circuit comprised of a Masterflex roller pump (Model 7553-70, Cole-Parmer, Vernon Hills, Ill.), compliance chamber to assure steady flow, parallel-plate flow chamber (Cytodyne, La Jolla, Calif.; see Frangos, J., L. Mcintire, and S. Eskin, Shear-Stress Induced Stimulation Of Mammalian-Cell Metabolism, Biotechnology And Bioengineering, 1988. 32(8): p. 1053-1060.) and a medium reservoir, which was equilibrated with a 5% CO₂ balance air mixture and placed in a 37° C. water bath, in series (FIG. 21, A). Glass slides containing polymer films seeded with endothelial cells were mounted to parallel-plate flow chambers (FIG. 21, B) and then introduced in the flow circuit to expose the films to well-defined laminar fluid flow parallel to the direction of the channels. PU films seeded with EC were exposed to shear stress for twenty minutes or one hour. The average wall shear stress resulting from steady laminar flow of a Newtonian fluid between two parallel plates can be estimated using the following equation derived for a smooth surface in a parallel-plate flow chamber, τ=6 Qμ/wh² (see Equation 2), where τ is shear stress in dynes/cm², Q is flow rate in ml/s, μ is the viscosity (1 cP), w is width of the parallel plates (2 cm) and, h is the spacing between the parallel plates in cm (0.0490 cm).

Visualization of cells. At the end of the experiment, cells were fixed in 4% paraformaldehyde (Sigma-Aldrich, St. Louis, Mo.) for 10 minutes and then cover-slipped with mounting medium containing DAPI (Vector Laboratories, Burlingame, Calif.). Images were acquired using a Zeiss Axiophot microscope (Zeiss, Thornwood, N.Y.) coupled to a CCD camera, with a 5× objective. Fluorescent micrographs were taken of the center portion of the film for each sample along with a corresponding bright field image to visualize channel outlines. The cells in eight randomly selected images per film were counted manually using a transparency of bright field image as a template. For unpatterned PU, the total number of cells in one image (1.17 mm²) was counted. For each image of cells on patterned PU, cells were counted separately on 5 plateau and 5 channel regions. Projected areas for these regions were 0.46±0.01 and 0.51±0.01 mm², for the plateaus and channels, respectively. Sides of the channels were included in the channel cell and area measurements. Statistical analysis was conducted using JMP-in software (SAS Institute Inc., Cary, N.C.). Difference in cell density between groups was determined using ANOVA followed by a Tukey-Kramer's HSD post-hoc test with the alpha level set at 0.05.

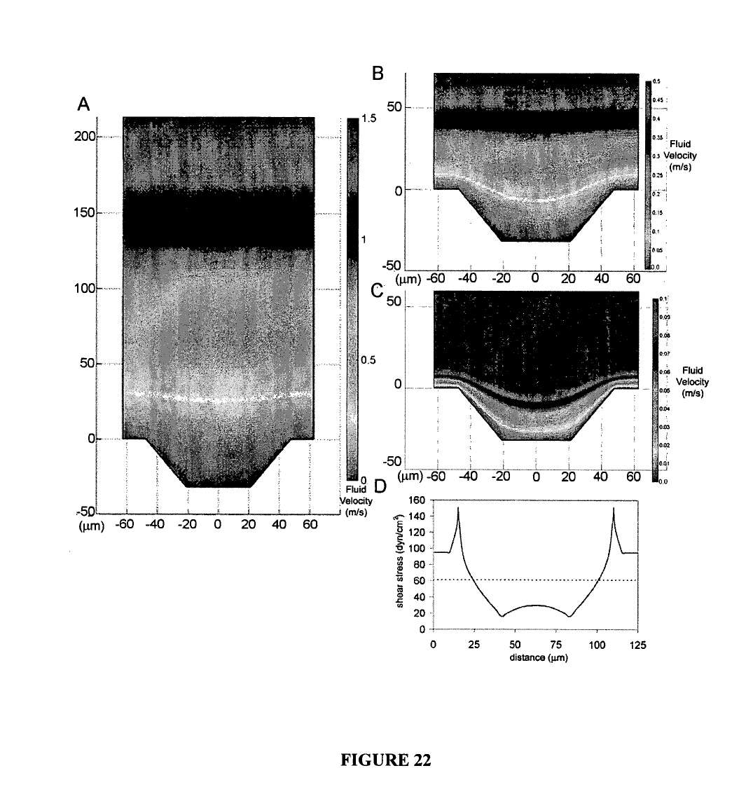

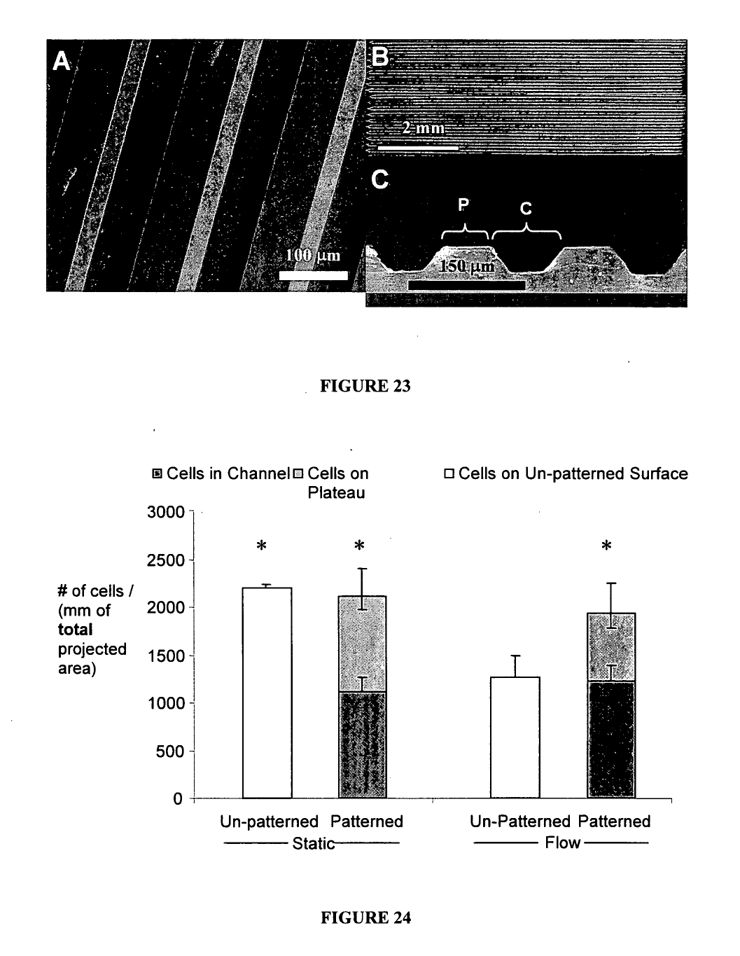

Computational fluid dynamic (CFD). CFD was used to estimate the shear stress in the channel and plateau regions. A 3-D model volume was created in pro/ENGINEER (2001, Parametric Technology Corporation) corresponding to the region shown in FIG. 22, A. The model volume had a height of 245 μm high (half the total spacing, h) and was applicable for regions where entry/exit effects are negligible (e.g., not near the triangular ends of the channel visible in FIG. 23, B). The maximum fluid velocity was prescribed to the plane at the top of the model geometry, corresponding to a total flow rate of 275 ml/min through the entire flow chamber. Boundary conditions were no-slip for the solid surfaces (e.g., the polymer surface), prescribed velocity for the top surface, straight-out (open to fluid flow) for the front and rear faces, and symmetric for both side surfaces (to eliminate false edge effects). A finite element mesh was automatically generated in FEMLAB (a computational fluid dynamics program also referred to as COMSOL-FemLab) and the velocity profile was determined for the model volume by solving the Navier-Stokes equations using FEMLAB's Chemical Engineering Module. From the velocity profile, the shear stress was calculated with the equation τ=−μ*dv/dy by determining the distance from the surface (dy) at which the velocity 0.015 m/s (dv).

b. Results

Characterization of silicon wafer templates and PU films. SEM images of channels in a silicon wafer are shown in FIG. 23, A and B. Wet etching attacks silicon preferentially in the 100 plane, producing a characteristic anisotropic V-shaped etch, with sidewalls that form a 54.7° angle with the surface (35.3° from the normal). Since the silicon wafer is a negative impression of the cast PU films, the channels in the PU correspond to the embossed ridges on the silicon wafer surface (FIG. 23, A). The film surface was composed of 4 arrays of channels with each array comprised of a 4 mm×5 mm rectangle with 31 channels/rectangle (FIG. 23, B). Each channel is 32 microns deep, 42 microns wide at its base and 95 microns wide at it top (i.e., plateau-to-plateau distance). Solution casting afforded a simple, reproducible method of transferring the pattern from the silicon wafer to a PU film surface. Under the casting conditions used, films of approximately 160 μm in thickness were obtained. Once the PU films were peeled from the silicon wafer, they retained the complementary micro-patterned features of the silicon template as evidenced by the SEM image of a cross-section of a PU film in FIG. 23, C. For sake of simplicity, the channel floor along with its pair of sloping walls will be hitherto referred as the channel and the region between two adjacent channels as plateau.

Cell densities under static conditions. BAEC cultured for 24 hours on all PU substrates exhibited cobblestone morphology, typical of endothelial cells in a confluent monolayer (not shown). Cells adhered to all exposed surfaces, including the angled channel walls. Under static conditions, the total cell densities for patterned or un-patterned PU were similar, 2113±292 cells/mm2 or 2206±37 cells/mm², respectively (Table 2; FIG. 24). The local cell densities based on projected area in channels and plateaus were also similar (2121±287 and 2104±298 cells/mm², respectively), indicating that in static cultures, the presence of the micro-patterned features did not affect the total number of cells or their spatial distribution.

TABLE 2

| | Un-patterned PU | Patterned PU | Patterned PU | Patterned PU |
|---|---|---|---|---|
| | (total area) | (total area) | (plateau area) | (channel area) |
| Static | 2206 ± 37 | 2113 ± 292 | 2104 ± 298 | 2121 ± 287 |
| | (n = 3) | (n = 3) | (n = 3) | (n = 3) |
| Flow | 1269 ± 219 | 1951 ± 464 | 1490 ± 328 | 2345 ± 320 |
| | (n = 3)* | (n = 6) | (n = 6)* | (n = 6) |

For Table 2, cell densities (cells/mm²) are based on total and regional areas for un-patterned and patterned surfaces. The cell densities based on total area are calculated by dividing the total number of cells—regardless of whether they are in a plateau or channel—by the total area. The cell densities based on regional areas are calculated by dividing the cell numbers in a given a defined region (e.g., 5 plateaus or 5 channels) by the area of that region. Asterisks (*) represent significance difference in cell densities between flow and static conditions.

Shear-stress dose response. Patterned PU substrates (n=2 for each shear stress) were subjected to 10, 20, 40 or 60 dynes/cm2 for 20 minutes. With shear stresses up to 40 dynes/cm², minimal loss of cells occurred on the patterned surface (not shown). With 60 dynes/cm², some cell loss from the plateaus was apparent. Based on these observations, a shear stress of 60 dynes/cm² was employed in the remainder of this study.

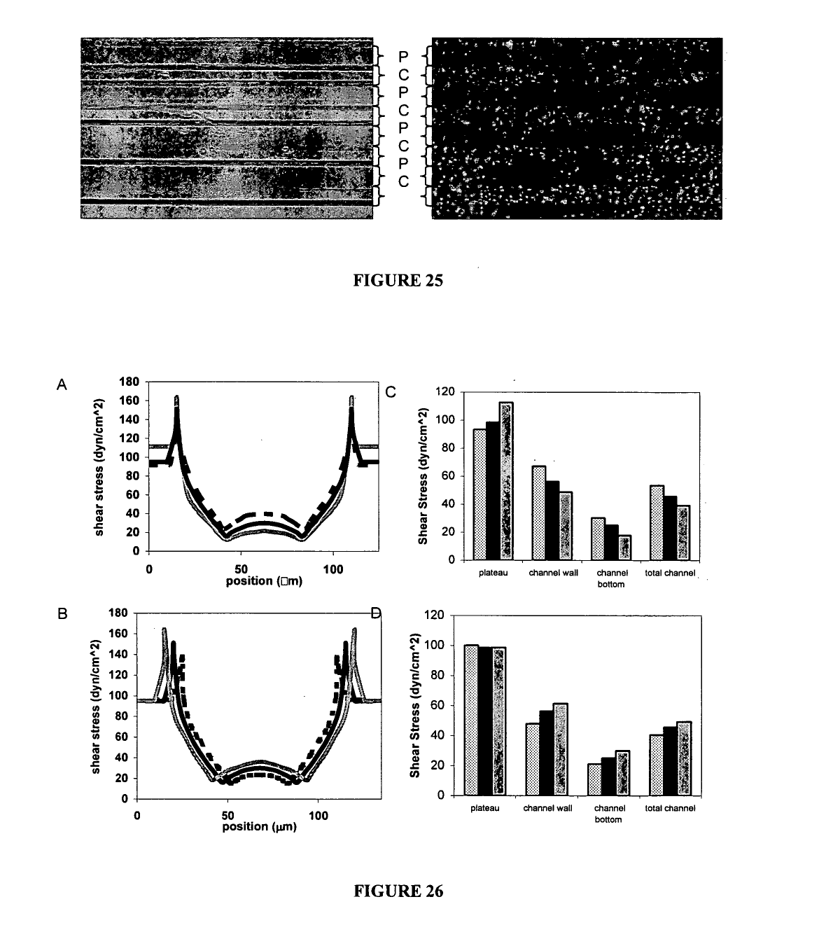

Effects of micropatterning on BAEC retention. Endothelial cells on patterned and un-patterned PU were exposed to 60 dynes/cm² for 1 hr. The number of cells remaining after flow, were determined and the corresponding cell densities were expressed in terms of total projected area (i.e., the total area of the PU surface quantified) and local projected areas (i.e., areas of channel and plateaus). In unpatterned PU exposed to flow, some regions were completely denuded of cells and the total cell density was decreased by 42% (FIG. 24, α<0.005). In contrast, when patterned PU was subjected to the same flow, the total cell density only decreased by 8% (FIG. 24, α>0.05). This improved cell retention was further investigated by considering the cell densities on the channels and plateaus separately. Exposure to flow detached cells from plateaus; overall local cell densities decreased by 29% (α<0.05) with smaller denuded regions (FIG. 25). Denuded regions of cells were not seen within the channels. Cell densities within in channels remained the same or even increased slightly after exposure to flow (Table 2, FIG. 24, 11% increase in cell density is not significant). Micropatterning the surface increased the total number of cell retained after flow, primarily by increasing the number of cells retained within the channels (FIG. 24).

Simulation of flow in channels in patterned PU surfaces. The 3-D model channel geometry used in the simulation studies was converted into a finite element mesh with 611 nodes and 2206 elements for the channel model. The velocity profiles for fluid flow in both the model patterned surface (FIG. 22, A-C) and the model unpatterned surface (not shown) were determined using FEMLAB. The solution was determined from 0-10 seconds, and found to not vary with time, indicating the solution had reached steady state. From the velocity profile, the shear stress was calculated for the patterned and un-patterned surfaces (FIG. 22, C, solid line and dotted horizontal line, respectively). The average shear stress for the entire channel (45.8 dyn/cm²) was 27.6% less than the average shear stress of the unpatterned surface (63.2 dyn/cm²) with the average shear stress on the channel floor (25.1 dyn/cm²) reduced more than that of the channel side-walls (56.3 dyn/cm²). The average shear stress of the plateau regions (98.5 dyn/cm²) was 55.6% greater than the shear stress of the control model, while the average stress on the side-walls of the channel was only somewhat reduced (10.9%). The average shear stress over the entire modeled patterned surface including the plateau and channel was 61.1 dyn/cm², similar to the average of the un-patterned surface (63.2 dyn/cm²).

Computational fluid dynamics (CFD) offers the potential to rapidly explore the effects of changing channel geometry and shape and thus could aid in the rational design of patterned surfaces. For instance, using the same fluid boundary conditions and fluid velocity, models were created that investigated the effects of increasing or decreasing overall channel depth (FIG. 26, A and C) and width (FIG. 26, B and D). The depth of the channel was varied from that of the physical model (32 μm, FIG. 26, A, black line), by either increasing (40 μm, FIG. 26, A, gray line), or decreasing (24 μm, FIG. 26, A, dashed line) by 8 μm or 25% of the original depth, without altering the width of either the overall channel, or the bottom surface. Increasing the channel depth, and the associated plateau-channel junction angle, increased both the peak shear stress and average plateau shear stress, while decreasing the shear stress throughout the channel (FIG. 26, C). To further investigate the effects of channel geometry, the width of the bottom surface of the channel was varied from the actual width of the physical model (42 μm, FIG. 26, B, black line), by either increasing (52 μm, FIG. 26, B, gray line) or decreasing (32 μm, FIG. 26, B, dashed line) by 10 μm, without altering any other aspect of the geometry or the fluid flow conditions. Increasing the channel width increased the peak shear stress at the plateau-channel junction but not the average plateau shear stress, while increasing the shear stress throughout the channel (FIG. 26, D).

C. Discussion

Without wishing to be bound by theory, it is believed that the concept that endothelial cells within local micro-domains with reduced flow-induced shear are more resistant to flow-induced cell loss explains the observations of others that when elevated transmural pressures is used to seed endothelial cells onto a graft, which forces some of the cells up to 50 microns into pores in the graft, the retention of cells following exposure to flow is increased. See, e.g., Chan, B. P., et al., In vivo performance of dual ligand augmented endothelialized expanded polytetrafluoroethylene vascular grafts, J Biomed Mater Res B Appl Biomater, 2005, 72(1): p. 52-63.

EC retention was increased in channels, which were exposed to reduced shear stress relative to unpatterned surfaces subjected to the same bulk flow rate. Importantly, this improvement in the channels did not come at the expense of decreased EC retention on the plateaus relative to unpatterned surface, despite an increase in shear stress on the plateaus. Without wishing to be bound by theory, it is believed that one possible reason that endothelial retention on the plateaus relative to the unpatterned surfaces was not decreased and was possibly even increased is that patterning appears to have prevented larger patches of denudation, which tended to occur on unpatterned surfaces. The presence of the protected channels prevented the propagation of denuded patches and thereby limited the cell loss in the plateaus by this mechanism. By improving EC retention in the channels, micro-patterning increased the total number of EC retained on the graft surface.

The remaining EC are preferentially subjected to reduced shear stress, which can diminish their production of anti-thrombogenic compounds. Diamond, S. L., S. G. Eskin, and L. V. McIntire, Fluid flow stimulates tissue plasminogen activator secretion by cultured human endothelial cells. Science, 1989, 243(4897): p. 1483-5; and Gooch, K. J. and J. A. Frangos, Flow- and bradykinin-induced nitric oxide production by endothelial cells is independent of membrane potential, Am J Physiol, 1996, 270(2 Pt 1): p. C546-51. It has been demonstrated, however, that EC can have positive effects on vessel health even when seeded on the adventitial surface of a vessel and thus not in contact with any flow. Nugent, H. M. and E. R. Edelman, Endothelial implants provide long-term control of vascular repair in a porcine model of arterial injury, J Surg Res, 2001, 99(2): p. 228-34.

Further, by using the present techniques, surface patterns that can significantly reduce the total shear force exerted over the entire luminal surface can be rapidly optimized for any conduit geometry envisioned. The effects of changes on shear stress distributions can be rapidly assessed using CFD, and the ability of promising designs to support EC retention can then be evaluated experimentally. Specifically, a simple force balance on the volume of fluid in a conduit of constant diameter dictates that the shear force exerted along the walls of the conduit must balance the pressure drop times the cross-sectional area of the fluid. Since surface texture has only a negligible effect on the pressure drop of laminar flow through a conduit, the total shear force exerted on the luminal surface therefore is not affected by the micropatterning. A corollary to this statement is that if micropatterning decreases the total shear force applied to one set of regions (e.g., the channels), it must increase the total force applied to another (e.g., the plateaus). A modest decrease in average shear stress, however, is possible as micropatterning can increase the area over which the total shear force is applied. Without wishing to be bound by theory, it is believed that these considerations, coupled with the experimental data, indicate that the primary mechanism by which micropatterning improves EC retention is by lowering shear stresses in local regions and not average shear stress over the entire lumen surface.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A synthetic conduit comprising a substantially tubular body comprising substantially circumferential polymer fibers; wherein the body has an exterior surface, an interior surface, and a lumen extending therethrough; and wherein the body has microscale features disposed at the interior surface.

2. The conduit of claim 1, wherein the fibers are electrospun.

3. The conduit of claim 1, further comprising at least one smooth muscle cell, transfected fibroblast cell, or a mixture thereof adhered to the exterior surface.

4. The conduit of claim 1, further comprising at least one endothelial cell, nerve cell, Schwann cell, glial cell, stem cell, or a mixture thereof adhered to the interior surface.

5. The conduit of claim 1, wherein the polymer fibers comprise segmented polyurethane fibers.

6. The conduit of claim 1, wherein the polymer fibers are nonbiodegradable.

7. The conduit of claim 1, further comprising a supplementary material, wherein the supplementary material comprises at least one of collagen, fibrin, chitin, laminin, polyethylene glycol, a synthetic peptide, a polysaccaride, a proteoglycan, an extracellular matrix component, or a mixture thereof.

8. The conduit of claim 1, further comprising at least one additive, wherein the additive comprises a pharmaceutically active agent, an antithrombogenic agent, or heparin.

9. The conduit of claim 1, wherein the microscale features are reservoirs, ridges, protrusions, grooves, or wells.

10. The conduit of claim 1, wherein the microscale features comprise ridges having an average width of from about 10 μm to about 500 μm and an average height of from about 20 μm to about 60 μm.

11. The conduit of claim 1, wherein the microscale features comprise grooves having an average width of from about 10 μm to about 500 μm and an average depth of from about 20 μm to about 60 μm.

12. The conduit of claim 1, wherein the microscale features comprise ridges or grooves, and wherein the features are disposed substantially parallel with the lumen.

13. The conduit of claim 1, wherein the substantially tubular body comprises at least one first layer of substantially circumferential electrospun polymer fibers and at least one second layer of polymer fibers, wherein the first layer is different from the second layer.

14. The conduit of claim 13, wherein the at least one second layer of polymer fibers is disposed inside the at least one first layer of substantially circumferential electrospun polymer fibers.

15. The conduit of claim 13, further comprising at least one third layer of polymer fibers, wherein the at least one first layer of substantially circumferential electrospun polymer fibers is disposed between the at least one second layer of polymer fibers and the at least one third layer of polymer fibers.

16. The conduit of claim 2, wherein the substantially tubular body comprises at least one first layer of substantially circumferential polymer fibers and at least one second layer of polymer fibers, wherein the first layer is different from the second layer.

17. The conduit of claim 16, wherein the at least one second layer of polymer fibers is disposed inside the at least one first layer of substantially circumferential polymer fibers.

18. The conduit of claim 16, further comprising at least one third layer of polymer fibers, wherein the at least one first layer of substantially circumferential electrospun polymer fibers is disposed between the at least one second layer of polymer fibers and the at least one third layer of polymer fibers.

19. A vascular prosthesis comprising a substantially tubular body comprising substantially aligned, substantially circumferential, electrospun polyurethane fibers;
wherein the body has an exterior surface, an interior surface, and a lumen with a diameter of from about 2 mm to about 4 mm extending therethrough;
wherein the body has microscale grooves disposed at the interior surface substantially parallel to the lumen;
wherein the grooves have an average width of from about 50 μm to about 100 μm and an average depth of from about 20 μm to about 60 μm;
wherein at least one smooth muscle cell is adhered to the exterior surface; and
wherein at least one endothelial cell is adhered to the interior surface.

20. A nerve regeneration scaffold comprising a substantially tubular body comprising substantially aligned, substantially circumferential, electrospun polyurethane fibers;
wherein the body has an exterior surface, an interior surface, and a lumen with a diameter of from about 2 mm to about 4 mm extending therethrough;
wherein the body has microscale ridges or grooves disposed at the interior surface substantially parallel to the lumen; and
wherein at least one transfected fibroblast cell is adhered to the exterior surface.

21. The nerve regeneration scaffold of claim 20, further comprising at least one nerve cell, Schwann cell, glial cell, stem cell, or a mixture thereof adhered to the interior surface.

22. A method of preparing a synthetic conduit comprising the step of spinning a polymer onto a rotating mandrel bearing microscale features, thereby providing a substantially tubular body comprising substantially circumferential polymer fibers; wherein the body has an exterior surface, an interior surface, and a lumen extending therethrough; and wherein the body has complementary microscale features disposed at the interior surface.

23. The method of claim 22, further comprising the step of removing the body from the mandrel.

24. The method of claim 22, wherein the spinning step is electrospinning.

25. The method of claim 22, further comprising the step of co-electrospinning a supplementary material onto the rotating mandrel.

26. The method of claim 24, further comprising the step of co-spinning a supplementary material onto the rotating mandrel.

27. The method of claim 22, wherein the supplementary material comprises at least one of collagen, fibrin, chitin, laminin, polyethylene glycol, a synthetic peptide, a polysaccaride, a proteoglycan, an extracellular matrix component, or a mixture thereof.

28. The method of claim 22, further comprising adhering at least one smooth muscle cell, transfected fibroblast cell, or a mixture thereof to the exterior surface.

29. The method of claim 22, further comprising adhering at least one endothelial cell, nerve cell, Schwann cell, glial cell, stem cell, or a mixture thereof to the interior surface.

30. A method of preparing a vascular prosthesis comprising the steps of:
a. electrospinning a solution of polymer onto a rotating mandrel bearing microscale features, thereby providing a substantially tubular body comprising substantially circumferential electrospun polymer fibers; wherein the body has an exterior surface, an interior surface, and a lumen extending therethrough; and wherein the body has complementary microscale features disposed at the interior surface;
b. adhering at least one smooth muscle cell to the exterior surface; and
c. adhering at least one endothelial cell to the interior surface.

31. A method of implanting a vascular prosthesis comprising the steps of:
a. providing the prosthesis produced by the method of claim 30; and
b. implanting the prosthesis into a subject.

32. A method of preparing a nerve regeneration scaffold comprising the steps of:
a. electrospinning a solution of polymer onto a rotating mandrel bearing microscale features, thereby providing a substantially tubular body comprising substantially circumferential electrospun polymer fibers; wherein the body has an exterior surface, an interior surface, and a lumen extending therethrough; and wherein the body has complementary microscale features disposed at the interior surface; and
b. adhering at least one transfected fibroblast to the exterior surface.

33. The method of claim 32, further comprising the step of:
c. adhering at least one nerve cell, Schwann cell, glial cell, stem cell, or a mixture thereof to the interior surface.

34. A method of implanting a nerve regeneration scaffold comprising the steps of:
a. providing the scaffold produced by the method of claim 32; and
b. implanting the scaffold into a subject.
